(19) United States
(12) Patent Application Publication
(10) Pub. No.: US 2023/0159597 A1
(43) Pub. Date: May 25, 2023

(54) LIPID-PEPTIDE FUSION INHIBITORS AS SARS-COV-2 ANTIVIRALS

(71) Applicant: Wisconsin Alumni Research Foundation, Madison, WI (US)

(72) Inventors: Matteo POROTTO, New York, NY (US); Anne MOSCONA, New York, NY (US); Samuel GELLMAN, Madison, WI (US); Victor OUTLAW, Madison, WI (US); Zhen YU, Madison, WI (US)

(21) Appl. No.: 17/996,917

(22) PCT Filed: Apr. 22, 2021
(86) PCT No.: PCT/US21/28667

§ 371 Date: Oct. 21, 2022

Related U.S. Application Data
(60) Provisional application No. 63/015,479, filed on Apr. 24, 2020.

(51) Int. Cl. C07K 14/005; A61P 31/14; A61K 47/60; A61K 47/54

(52) U.S. Cl. CPC C07K 14/005; A61P 31/14; A61K 47/60; A61K 47/543; A61K 47/545; C07K 2319/10; A61K 38/00

(57) ABSTRACT

Described herein is a composition and method of treating COVID-19 with lipid-peptide fusion antiviral therapy. Also described is a composition and method of treating Ebola with lipid-peptide fusion antiviral therapy.

### CROSS-REFERENCES TO RELATED APPLICATIONS

This application claims the benefit of priority under 35 U.S.C. § 119(e) of U.S. Provisional Application No. 63/015,479, filed Apr. 24, 2020, the contents of which are hereby incorporated by reference in its entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT

This invention was made with government support under grants AI114736 and AI121349 awarded by the National Institutes of Health. The government has certain rights in the invention.

### BACKGROUND OF THE INVENTION

Infection by coronaviruses, including the Severe acute respiratory syndrome virus SARS-CoV-2 (COVID) virus, requires membrane fusion between the viral envelope and the lung cell membrane. The fusion process is mediated by the virus's envelope glycoprotein, also called spike protein or S. No therapeutic options are currently available for the prophylaxis or treatment of infected individuals. The newly emerged pathogenic virus SARS-CoV-2 (the cause of COVID-19 respiratory disease) represents a worldwide threat to human health and social order. Therefore, given the current pandemic of COVID-19, the development of an effective antiviral therapy against these coronaviruses, especially SARS-CoV-2, is of highest priority not only nationally but also worldwide.

### SUMMARY OF THE INVENTION

In certain aspects, the invention provides a peptide including or with SEQ ID:NO2 or SEQ ID NO:3. In certain aspects, the invention provides a peptide including or with a sequence with more than 80%, 85%, 90%, 95%, but less than 100% homology with SEQ ID NO:1, SEQ ID NO:2 or SEQ ID NO:3.

In certain aspects, a SARS lipid-peptide fusion includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, and a lipid tag.

In some embodiments, the lipid tag is Cholesterol, Tocopherol, or Palmitate.

In certain aspects, a SARS lipid-peptide fusion inhibitor includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, and a spacer.

In some embodiments, the spacer is a polyethylene glycol (PEG). In some embodiments, the spacer is PEG4. In some embodiments, the lipid tag is Cholesterol, Tocopherol, or Palmitate.

In some embodiments, the SARS lipid-peptide fusion inhibitor further includes a cell penetrating peptide sequence (CPP). In some embodiments, the CPP is HIV-TAT.

In certain aspects, a pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, and a pharmaceutically acceptable excipient.

In certain aspects, a pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, a pharmaceutically acceptable excipient.

In some embodiments, the lipid tag is Cholesterol, Tocopherol, or Palmitate.

In certain aspects, a pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, a spacer, and a pharmaceutically acceptable excipient.

In some embodiments, the spacer is a polyethylene glycol (PEG). In some embodiments, the spacer is PEG4. In some embodiments, the lipid tag is Cholesterol, Tocopherol, or Palmitate.

In some embodiments, the coronavirus lipid-peptide fusion inhibitor further includes a cell penetrating peptide sequence (CPP). In some embodiments, the CPP is HIV-TAT.

In certain aspects, a SARS-COV-2 (COVID-19) antiviral composition includes a SARS-COV-2 (COVID-19) lipid-peptide fusion inhibitor and a pharmaceutically acceptable excipient. The SARS-COV-2 (COVID-19) lipid-peptide fusion inhibitor further includes a peptide selected from SEQ ID NO:1, SEQ ID NO:2 and SEQ ID NO:3, a lipid tag, a spacer, and a CPP.

In some embodiments, the peptide is SEQ ID NO:2 or SEQ ID NO:3.

In certain aspects, the invention provides a method of treating COVID-19 that includes administering to a patient an antiviral pharmaceutical composition. The antiviral pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, a spacer, a CPP, and pharmaceutically acceptable excipients.

In some embodiments, the lipid tag is Cholesterol, Tocopherol, or Palmitate.

In some embodiments, the antiviral pharmaceutical composition is administered per airway or subcutaneously. In some embodiments, the antiviral pharmaceutical composition is administered intranasally. In some embodiments, the antiviral pharmaceutical composition is administered as nasal drops or a spray.

### BRIEF DESCRIPTIONS OF THE DRAWINGS

FIG. 1: The S(Spike) protein. Repeat sections HRN and HRC at either end recognize each other, and snap together to form the folded structure. Fusion inhibitory peptides bind to the repeat section and prevent formation of the folded structure, therefore blocking viral fusion and entry.

FIG. 2: Infection and Cell-entry by coronaviruses.

FIG. 3: Ebola viral entry via the endosome pathway. From Falzarano D, Feldmann H. Virology. Delineating Ebola entry. Science 2015.

FIG. 4: Modular design of SARS-CoV-2 inhibitors derived from the viral envelope spike (S) protein.

FIG. 5: Lipid modified HRC peptides block both early and latent coronavirus viral entry. This is a schematic representation of results obtained using our lipid-conjugated MERS-derived peptides. Figure from Park and Gallagher, Lipidation increases antiviral activities of coronavirus fusion-inhibiting peptides, Virology 2017; 511, 9-18.

FIG. 6: Fusion inhibition assay of MERS-CoV-S peptides on MERS-S mediated fusion.

FIG. 7: Therapeutic efficacy. Mice (N=5/group) were treated with a single dose of 2 mg/Kg i.p. of cholesterol tagged or untagged peptide (or untreated) 16 hrs after infection with 10 LD50 MERS-CoV (10² TCID₅₀/per mouse i.n.). Note that if given intranasally before infection, even untagged peptide is 100% protective.

FIG. 8: TAT-EBOLA-dPEG4-Toc protects mice from the lethal (MA-)ZEBOV infection. 5-6 weeks old BALB/c mice received intraperitoneal challenge of (MA-)ZEBOV 24 hr after the first peptide treatment, and were followed for 5 weeks post infection. Peptide (10 mg/kg dissolved in isotonic water) was administered intraperitoneally daily for 15 days.

FIG. 9: Intracellular localization of TAT and lipid-conjugated peptides. Vero cell monolayers were incubated for 60′ at 37° C. with 10 μM of the indicated peptides. Cells were fixed, permeabilized with 0.02% Tween-20 in PBS, stained with custom made biotin-conjugated antipeptide antibodies. The anti-peptide antibodies were detected with streptavidin-phycoerythrin (PE). Cells were counterstained with DAPI (Nuclei staining). PE (emission 580 nm) and DAPI (emission 460 nm) fluorescence was acquired.

FIG. 10: Design of HRC derived C-peptides and sequence. Software (http://www.uniprot.org/align/) indicates the similarity of each HRN target to that of MERS-CoV. The residues that interact with C-peptides are highlighted in bold font and residues located at non-interacting regions are shaded in gray.

FIG. 11: Lipid tagged from SARS-CoV-2 S. C-peptides derived from the SARS-CoV-2 S HRC region will be synthesized. In the third row (DISG . . . QEL) is the sequence that we recently tested and compared to EK1 peptide.

FIG. 12: Crystal structure of the 6HB assembly formed by the HRC and HRN domains of the SARS-CoV-2 S protein (PDB 6LXT). In HRC, note central helix and extended segments on either side.

FIG. 13: Sequence of the HRC domain of the SARS-CoV-2 S protein (top), with numbering shown at each end, as represented in D-1. The two “h” symbols indicate the boundaries of the helical segment. D-2 contains two α-amino acid residue changes (red), to optimize the ion pairing array. D-3 corresponds to the HRC domain of MERS, and D-4 is the peptide EK1, derived from the MERS HRC (changes in red).

FIG. 14: Fusion inhibition assays show that MERS-CoV-S C-peptides block SARS-CoV-2-S mediated fusion. Peptide aa sequence is shown. A control peptide (parainfluenza sequence) is shown in black.

FIGS. 15A-15B: Plaque reduction assays. FIG. 15A: 100% reduction in SARS-CoV-2 infection was observed using live virus and our MERS lipid-peptide in cell culture.

FIG. 15B. Plaque inhibition assay for the EBO-fusions. Peptides were serially diluted 10-fold in sterile water (10 uM thru 0.005 uM), each peptide dose was mixed with an equal volume of virus containing 500PFU/mL diluted in MEM, and the peptide/virus mixtures were incubated at 37 C for 1 hour. Each peptide dose/virus mixture was inoculated onto triplicate wells of Vero E6 cells in 6-well plates (0.2-mL per well) and allowed to adsorb for 1 hour at 37C. Cell monolayers were rinsed twice with PBS prior to addition of medium overlay containing MEM, 5% fetal bovine serum, antibiotics, and ME agarose (0.6%). Cultures were incubated at 37 C for 6 days, overlaid with medium containing neutral red as a stain, and plaques were counted 24-48 hours later. Virus controls were mixed with sterile water instead of peptides.

FIG. 16: Inhibition of SARS CoV-2 glycoprotein fusion with SARS and MERS peptides. The SARS peptide has an IC50 of around 6 nm with ACE2 and only 0.09 nM without ACE2.

FIG. 17: Inhibition of SARS CoV-2 glycoprotein fusion with the indicated peptides.

FIG. 18: Sequence of the indicated peptides used in FIG. 17.

FIG. 19: Inhibition of SARS CoV-2 glycoprotein with the indicated proteins.

FIG. 20: Inhibition of viral infection with the SARS-CoV-2 peptide.

FIG. 21: The human airway epithelium (HAE).

FIG. 22: Human parainfluenza-GFP in HAE over time (3 days).

FIG. 23: Human airway epithelium (HAE) Infection with SARS-CoV-2 bearing EGFP.

FIG. 24: Human lung organoids infected with parainfluenza virus bearing EGFP.

FIG. 25: In vivo efficacy vs. Nipah (lethal virus) infection in golden hamsters demonstrates that 2 mg/kg/d subcutaneous delivery of the lipid-peptide was effective.

FIG. 26: In vivo efficacy vs. Nipah (lethal virus) infection in golden hamsters: the lipid-peptide was administered intranasally. An administration at 1 day before, day of, 1 day after can provide 60% protection from lethal infection.

FIG. 27: In vivo efficacy vs. influenza infection. Peptides given intranasally three times:1 day before, day of, 1 day after 1000x lower viral titer in cotton rats.

FIG. 28: In vivo efficacy for preventing measles infection (fatal encephalitis) in mice with measles peptides. Both subcutaneous and intranasal administration were explored.

FIG. 29: Design of ferret studies, as in Kim et al.

### DETAILED DESCRIPTION OF THE INVENTION

The invention covers lipid-peptide molecules for the prevention and treatment of COVID-19. The invention uses designed peptides that block SARS-CoV-2 entry into cells and will likely prevent and/or abrogate infection in vivo and prevent transmission. The inventors discovered that that this type of lipid-peptide molecule is highly effective at preventing and even treating lethal infections of other viruses, like measles, lethal Nipah virus, influenza, and others. The designed peptides are highly effective at inhibiting live SARS-CoV-2 (COVID) virus infection in cultured cells and ex vivo.

Infection by coronaviruses, including the SARS-CoV-2 (COVID) virus, requires membrane fusion between the viral envelope and the lung cell membrane. The fusion process is mediated by the virus's envelope glycoprotein, also called spike protein or S. The inventors designed specific peptides, linked to lipids, that inhibit viral fusion and infection by binding to transitional stages of the spike protein, preventing its function. Importantly, based on evidence from the other viruses that the inventors targeted, these antivirals can be given by the airway, by nasal drops, are not toxic, and have good half-life in the lungs. The fact that they can be given via the nose and inhalation makes them feasible for widespread use.

The inventors designed several assays for assessing potency and mechanism in BSL2 laboratory conditions, which thus far precisely predict efficacy vs. live SARS-CoV-2 in cell culture. The prototype peptides are highly effective in blocking SARS-CoV-2 spike protein fusion and viral entry assays in cultured cells, and at inhibiting live SARS-CoV-2 (COVID) virus infection in vitro and ex vivo. Improvements to these antivirals will make them even more effective, more resistant to being broken down in the lungs or blood, and better at interacting with the spike protein to block its transitional states. Testing the lead antivirals in animal models will show utility for preventing and treating infection and preventing contagion from an infected animal to a healthy animal, including treatment as nasal drops or spray to prevent infection of healthcare workers.

In certain aspects, the invention provides a peptide including or with SEQ ID:NO2 or SEQ ID NO:3. In certain aspects, the invention provides a peptide including or with a sequence with more than 80%, 85%, 90%, 95%, but less than 100% homology with SEQ ID NO:1, SEQ ID NO:2 or SEQ ID NO:3.

In certain aspects, a SARS lipid-peptide fusion includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, and a lipid tag.

In some embodiments, the lipid tag is Cholesterol, Tocopherol, or Palmitate.

In certain aspects, a SARS lipid-peptide fusion inhibitor includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, and a spacer.

In some embodiments, the spacer is a polyethylene glycol (PEG). In some embodiments, the spacer is PEG4. In some embodiments, the lipid tag is Cholesterol, Tocopherol, or Palmitate.

In some embodiments, the SARS lipid-peptide fusion inhibitor further includes a cell penetrating peptide sequence (CPP). In some embodiments, the CPP is HIV-TAT.

In certain aspects, a pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, and a pharmaceutically acceptable excipient.

In certain aspects, a pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, a pharmaceutically acceptable excipient.

In some embodiments, the lipid tag is Cholesterol, Tocopherol, or Palmitate.

In certain aspects, a pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, a spacer, and a pharmaceutically acceptable excipient.

In some embodiments, the spacer is a polyethylene glycol (PEG). In some embodiments, the spacer is PEG4. In some embodiments, the lipid tag is Cholesterol, Tocopherol, or Palmitate.

In some embodiments, the coronavirus lipid-peptide fusion inhibitor further includes a cell penetrating peptide sequence (CPP). In some embodiments, the CPP is HIV-TAT.

In certain aspects, a SARS-COV-2 (COVID-19) antiviral composition includes a SARS-COV-2 (COVID-19) lipid-peptide fusion inhibitor and a pharmaceutically acceptable excipient. The SARS-COV-2 (COVID-19) lipid-peptide fusion inhibitor further includes a peptide selected from SEQ ID NO:1, SEQ ID NO:2 and SEQ ID NO:3, a lipid tag, a spacer, and a CPP.

In some embodiments, the peptide is SEQ ID NO:2 or SEQ ID NO:3.

In certain aspects, the invention provides a method of treating COVID-19 that includes administering to a patient an antiviral pharmaceutical composition. The antiviral pharmaceutical composition includes a peptide including or with SEQ ID:NO2 or SEQ ID NO:3, or a peptide including or with a sequence with more than 80%, 85%, 90%, 95%, but less than 100% homology with SEQ ID NO:1, SEQ ID NO:2 or SEQ ID NO:3, a lipid tag, a spacer, a CPP, and pharmaceutically acceptable excipients.

In some embodiments, the lipid tag is Cholesterol, Tocopherol, or Palmitate.

In some embodiments, the antiviral pharmaceutical composition is administered per airway or subcutaneously. In some embodiments, the antiviral pharmaceutical composition is administered intranasally. In some embodiments, the antiviral pharmaceutical composition is administered as nasal drops or a spray.

### Examples

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### EXAMPLE 1 General Concept

Coronavirus Infection

Coronaviruses (CoVs) can cause life-threatening diseases. The latest disease was recently named coronavirus disease 2019 (abbreviated “COVID-19”) by the World Health Organization. COVID-19 is caused by the coronavirus strain SARS-CoV-2. Like its predecessors SARS-CoV-1 and middle eastern respiratory syndrome virus MERS-CoV, SARS-CoV-2 is a betacoronavirus. No vaccines or treatments for COVID-19 are yet available. Antivirals that target viral entry into the host cell have been proven effective against a wide range of viral diseases.

Coronavirus Entry Pathway into Target Cells

Coronaviruses employ a type I fusion mechanism to gain access to the cytoplasm of host cells. Other pathogenic viruses that employ the type I fusion mechanism include HIV, paramyxoviruses and pneumoviruses. Merger of the viral envelope and host cell membrane is driven by profound structural rearrangements of trimeric viral fusion proteins; infection can be arrested by inhibiting the rearrangement process.

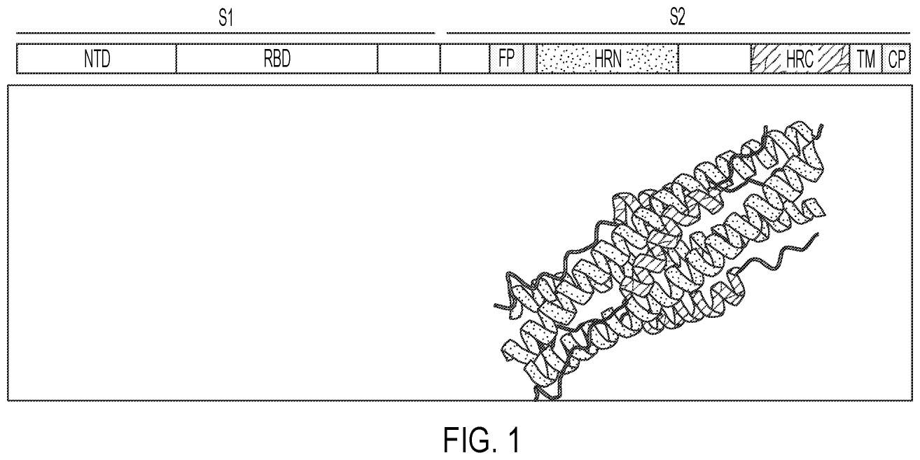

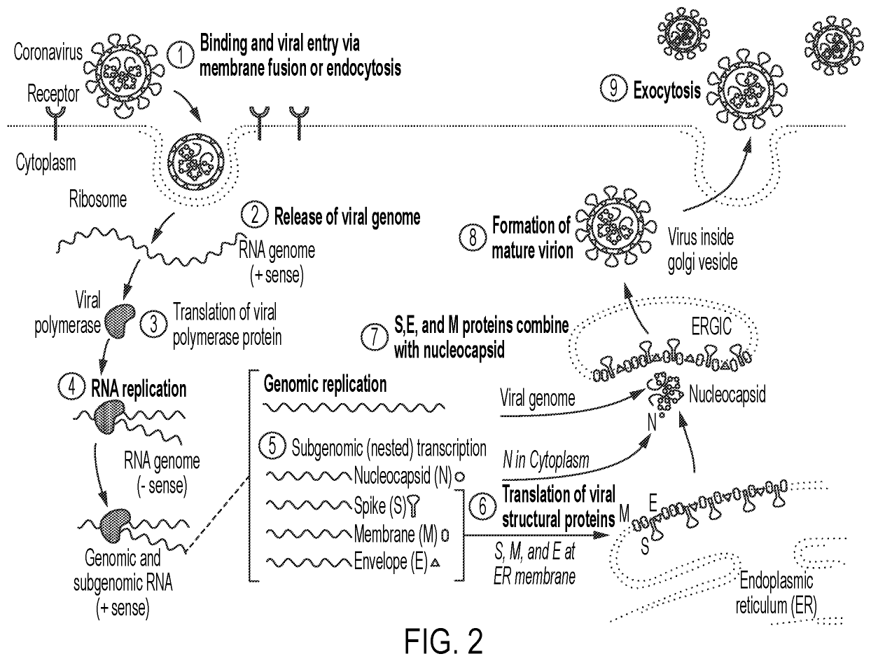

Infection by coronavirus requires membrane fusion between the viral envelope and the cell membrane. Depending on the cell type and the coronavirus strain, fusion can occur at either the cell surface membrane or in the endosomal membrane. The fusion process is mediated by the viral envelope glycoprotein (S), a —1200 residue heavily-glycosylated type-I integral membrane protein as a large homotrimer, each monomer having several domains (FIGS. 1, 2). A receptor binding domain (RBD) —distal to the viral membrane —is responsible for cell surface attachment. Membrane merger is mediated by a proximal cell fusion domain (FD). Concerted action by the RBD and FD is required for fusion. Upon viral attachment (and uptake in certain cases), host factors (receptors and proteases) trigger large scale conformational rearrangements in the FD, driven by formation of an energetically stable 6-helix bundle (6HB) that couples protein refolding directly to membrane fusion. The FD is thought to form a transient pre-hairpin intermediate composed of a highly conserved trimeric coiled-coil core that can be targeted by fusion inhibitory peptides (referred to as C-terminal heptad repeat, C-peptides, or HRC peptides).

Like the influenza HA, this S exists as a trimer on the virion surface and mediates attachment, receptor binding and membrane fusion. The betacoronaviruses S proteins' host cell receptors identified thus far include angiotensin-converting enzyme 2 (ACE2) for SARS-CoV-1 and dipeptidyl peptidase-4 (DPP4) for MERS-CoV. SARS-CoV-2 was found to use the human angiotensin-converting enzyme 2 (hACE2) for entry (and may use other receptors as yet unknown). S undergoes cleavage by a host protease to generate S₁ and S₂. Priming with the receptor and cleavage are both necessary for membrane merger

Pathways of Viral Entry and Strategies for Inhibition

The activation step that initiates a series of conformational changes in the fusion protein leading to membrane merger differs depending on the pathway that the virus uses to enter the cell. For many paramyxoviruses, upon receptor binding, the attachment glycoprotein activates the fusion protein (F) to assume its fusion-ready conformation at the cell surface at neutral pH. We and others have shown that for these viruses (that fuse at the cell membrane), C-peptides derived from the HRC region of the fusion protein ectodomain inhibit viral entry with varying activity and that lipid conjugation markedly enhances their antiviral potency and simultaneously increases their in vivo half-life. By targeting lipid-conjugated fusion inhibitory peptides to the plasma membrane, and by engineering increased HRN-peptide binding affinity, we have increased antiviral potency by several logs. The lipid-conjugated inhibitory peptides on the cell surface directly target the membrane site of viral fusion. By adding poly-ethylene glycol (PEG) linkers (such as PEG4) to the compounds between the lipid moiety and the peptide, we further increased the broad spectrum activity and potency of the conjugates. For the purpose of this application, the words “linker” and “spacer” are used interchangeably. We demonstrated in vivo efficacy of lipid-conjugated fusion inhibitory peptides against lethal Nipah virus infection in golden hamsters and non-human primates, measles virus infection in mice and cotton rats, and human parainfluenza virus type 3 infection in cotton rats.

For viruses that do not fuse at the cell membrane the target for C-peptides is generally thought to be inaccessible. Example of these viruses are influenza and Ebola viruses. The fusion proteins of influenza (hemagglutinin protein; HA) and of Ebola (GP) are activated to fuse only after intracellular internalization. We showed that our lipid-conjugated peptides derived from influenza HA inhibit infection by influenza, suggesting that the lipid-conjugation-based strategy permits the use of fusion-inhibitory peptides for viruses that fuse in the cell interior. A second strategy that we adopted for influenza is the addition of HIV-TAT (a well known cell-penetrating peptide, CPP) to enhance inhibition of intracellular targets. With the combination of these two strategies, HA derived peptides are effective in vivo against human strains of influenza virus.

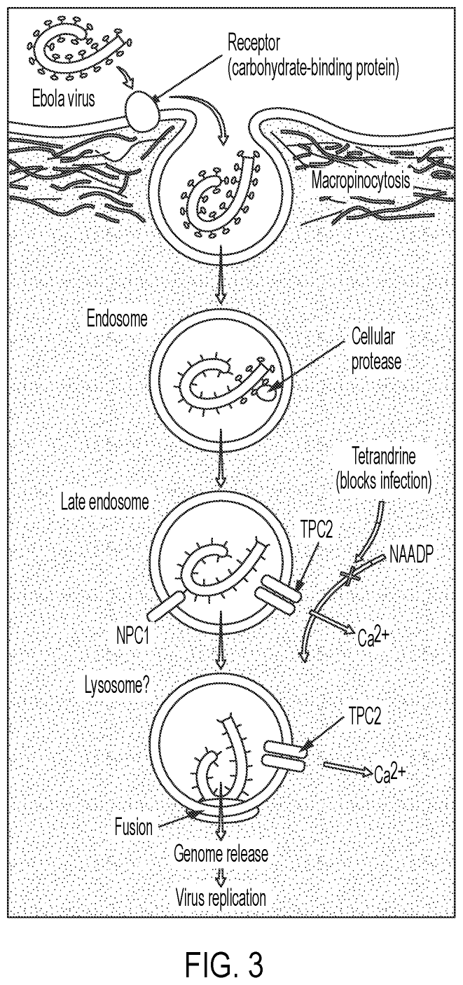

A similar strategy led to effective antiviral C-peptides for Ebola infection. In FIG. 3, the process of Ebola viral entry is depicted. The activation step leading to the Ebola GP₂ fusion occurs between the late endosome and the lysosome. In Ebola GP₂, the HRN and HRC regions are connected by a 25-residue linker, containing a CX₆CC motif and an internal fusion loop. Structural study of the fusion core of Ebola GP₂ led to the proposed use of GP₂ C-peptides as antivirals. However, the Ebola (EBOV)C-peptide showed low potency, in agreement with the notion that its target was accessible only in the endosome and not at the cell surface. Conjugation of the CPP HIV TAT to an Ebola fusion inhibitor (in an effort to enhance localization) improved its antiviral activity, resulting in an IC₅₀ value of about 5001 Based on this information, combined with our finding that lipid-conjugated influenza HA-derived peptides, we synthesized the Zaire (Z) EBOV GP₂-derived C-peptides described in Table 1. We have shown that polyethylene glycol (PEG) spacers inserted between the lipid moiety and the peptide leads to enhanced broad spectrum activity and potency. The lipid moieties and PEG4 spacers are located in the C-terminus of the C-peptides. In our influenza work, we found that addition of tocopherol moieties to the antiviral peptides increased the in vivo potency of the peptides, so we used tocopherol (Toc) in the design of anti-Ebola C-peptides.

Proof of Principle: Fusion Lipid Peptides

A major challenge in developing C-peptide fusion inhibitors for coronavirus may be that coronavirus viral entry can follow several entry pathways (FIG. 2). Some coronavirus strains can fuse at the cell surface, however several others initially endocytose, and fusion is triggered in the endosome. In some cases, the same strain, depending on the S cleavage site and the target host cell protease, can enter via different pathways. The virus can fuse on the cell surface or inside the cells.

For this reason, design of entry inhibitors for coronavirus is a challenge. We explored whether adding cell penetrating peptides and lipid moieties that promote endosomal localization would increase the antiviral potency.

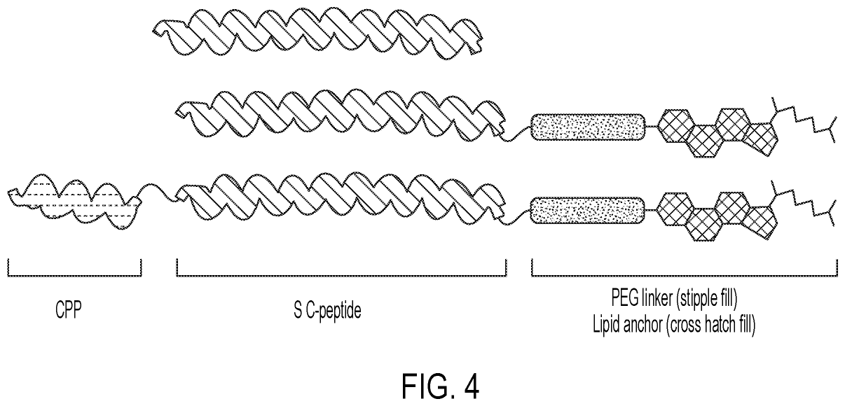

HRC peptides inhibit viral fusion and entry in a dominant-negative manner by binding to the pre-hairpin intermediate, preventing formation of the 6HB. For strains that fuse at the cell membrane (early entry), HRC peptides without additional components can prevent viral entry, but these peptides are ineffective on strains that fuse in the endosome (late entry). The intracellular sequestration of S could make it challenging to develop HRC peptide fusion inhibitors against endosomal fusing coronavirus strains. To target endosomal fusing coronaviruses including SARS-CoV-2, in addition to the proven lipidation and pegylation strategies, we incorporated a cell penetrating peptide sequence (CPP in FIG. 4) to further promote their endocytosis.

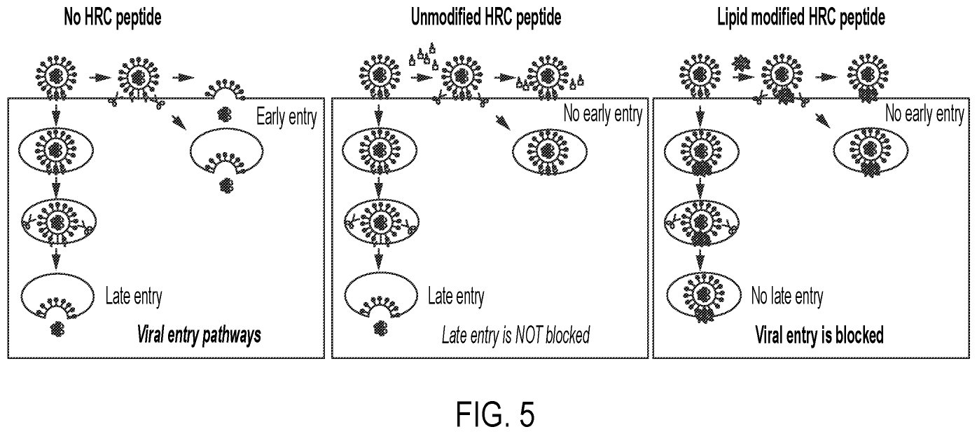

Earlier research on lipid-conjugated inhibitory peptides demonstrated that the lipid directs the peptide to cell membranes and increases antiviral efficacy. These conjugated peptides were shown, in published work, to inhibit both early and late entry strains of coronavirus (FIG. 5).

For viruses that fuse at the target cell membrane, lipid conjugation to HRC peptides markedly increases antiviral potency and in vivo half-life. Lipid conjugation also enables activity against viruses that do not fuse until they have been taken up via endocytosis. For example, we showed that lipid-conjugated HRC peptides derived from MERS (see below) inhibit MERS infection, suggesting that the lipid-conjugation-based strategy generates inhibitors of fusion with endosomal membranes. A similar strategy led to effective antiviral peptides for Ebola infection, which fuses between the late endosome and the lysosome. These lipid-peptides “follow” the virus into intracellular compartments.

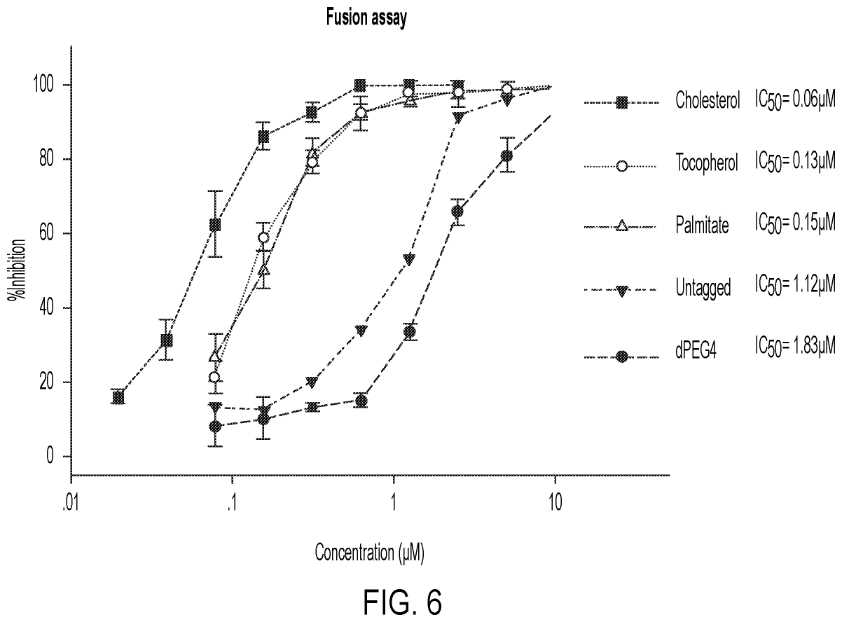

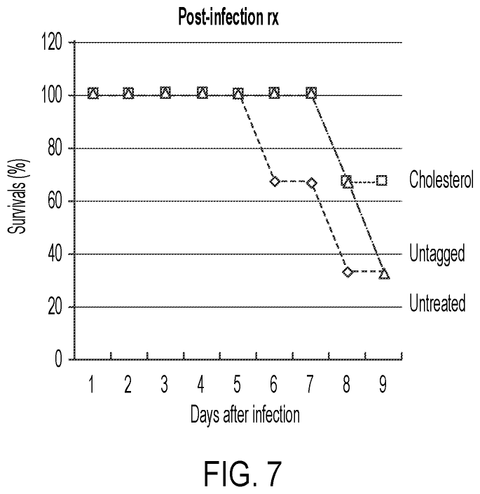

We designed and produced MERS-CoV-specific lipid-conjugated peptides based on a peptide sequence shown to be effective in vivo after intra-lung administration. In 2014, these peptides made by our design were tested against MERS-CoV in vitro (FIG. 6) and in vivo (FIG. 7). The lipid moieties increased the peptides' potency in fusion assays (FIG. 6) and increased their in vivo activity (FIG. 7). More recently, the Gallagher group has found that lipid-conjugation (using our peptides) increases antiviral potency of MERS-CoV-derived peptides up to 1000-fold, leading to CoV entry inhibition both at the plasma membrane and in endosomal compartments.

Proof of Principle: Inhibition of Live Ebola Infectivity In Vitro.

We compared the efficacy of the above C-peptides vs. live ZEBOV infection in vitro in collaboration with UTMB's BSL4 facility (Table 1). Control Ebola C-peptides derived from the same HR domain but without the TAT (CPP motif) sequence were ineffective even when lipid conjugated (100 μM was the highest concentration tested). Thus, the inhibitory activity for Ebola virus in particular requires both the TAT sequence and the lipid conjugation.

TABLE 1
EBOV ZAIRE GP derived peptides with lipid
modification inhibit live ZEBOV infection.
YGRKKKRRQRRR sequences correspond to the HIV
TAT sequence. Data from triplicate wells
repeated three times.

| | | Live ZEBOV | |
|---|---|---|---|
| | Sequence and | in vitro | |
| Name | modifications | [symbol] | [symbol] |
| EBOLA | [symbol] | >100 | >100 |
| EBOLA-dPEG4 | [symbol] | ~20 | >100 |
| EBOLA-dPEG4-Chol | [symbol] | ~10 | >100 |
| EBOLA-dPEG4-Toc | [symbol] | ~8 | 100 |
| TAT-EBOLA | [symbol] | ND | ND |
| TAT-EBOLA-dPEG4 | [symbol] | ~10 | >100 |
| TAT-EBOLA-dPEG4-Chol | [symbol] | ~10 | ~100 |
| TAT-EBOLA-dPEG4-Toc | [symbol] | ~0.8 | ~20 |

[symbol]

indicates data missing or illegible when filed

Proof of Principle: Inhibition of Mouse Adapted (MA)-ZEBOV In Vivo:

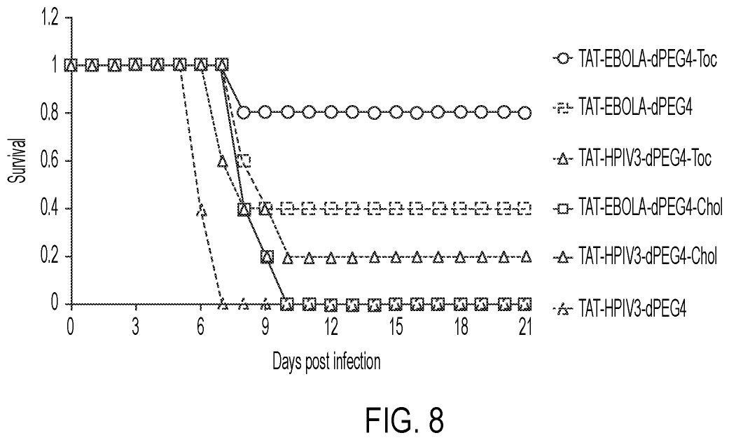

Three C-peptide inhibitors in Table 1 (highlighted in) were first tested for acute toxicity in mice at 20 mg/ml for 14 days by intraperitoneal (i.p.) delivery, without any tolerability issues. Mouse pharmacokinetic studies confirmed the presence of the lipid conjugated C-peptide inhibitors in the plasma for at least 24 hrs. For the in vivo study presented in FIG. 8, the animals were infected with 100_{LD50} of virus. We treated 5-6 week old BALB/c mice, 5 animals per group, with 10 mg/kg i.p. from day-1 to day 15 post-infection (˜100 μl isotonic aqueous solution per animal). All the infected animals showed a significant weight loss, however 4 out of the 5 animals that were treated prophylactically with the TAT-EBOLA-dPEG4-Toc peptide survived the infection (vs. none in the untreated group). The TAT-EBOLAdPEG4 without lipid partially protected 2 of 5 animals. None of the animals treated with the cholesterol conjugated C-peptides survived. The control group of animals were treated with HPIV3 C-derived peptides bearing the same TAT sequence, lipid moieties, and PEG linkers. The surviving animals were re-challenged and all the animals survived the second challenge, indicating that the treatment with C-peptide fusion inhibitors allowed for development of protective immunity.

Proof of Principle: Lipid-Conjugated Inhibitory Peptides Undergo Cellular Internalization.

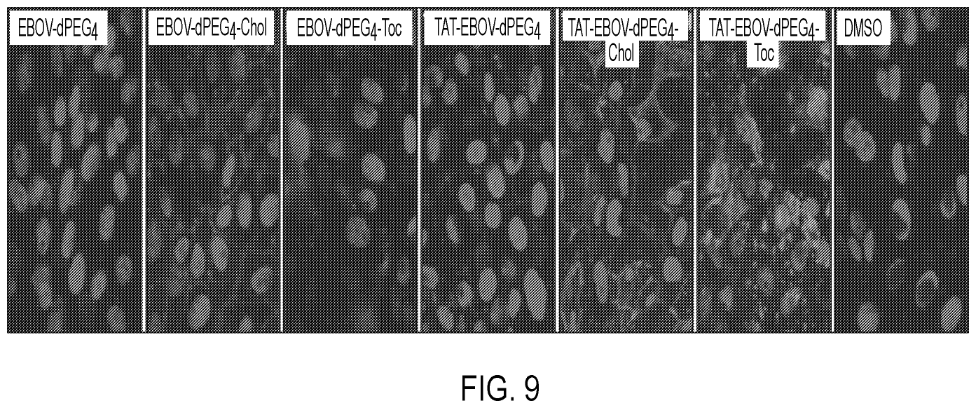

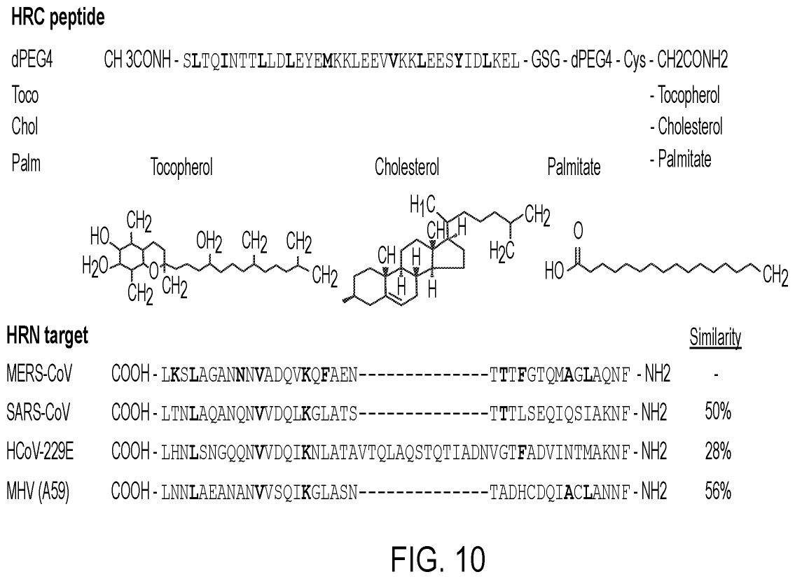

Since the TATEBOLA-dPEG4-Toc peptide is effective in vivo (FIG. 8), we asked whether its intracellular localization was different from the TAT-EBOLA-dPEG4-Chol (or other peptides), and analyzed cellular localization using confocal microscopy. The peptides (dissolved in DMSO to 1000 μM) were diluted in PBS to 10 μM, and added to live Vero cells at 37° C. Controls included peptides without lipids, and DMSO alone, and the peptides were detected with biotin-conjugated anti-peptide antibodies. The TAT-EBOLA-dPEG4-Toc treated cells show intense intracellular fluorescent spots. The EBOLA-dPEG4-Chol (without TAT) is mainly localized on the cell membrane with minimal cellular internalization; adding TAT increases the membrane localization and leads to partial intracellular localization. The TAT-EBOLA-dPEG4 was detected only at very low levels at the cell membrane and inside the cells compared to the lipid tagged peptides. FIG. 9 shows that the TAT-EBOLA-dPEG4-Toc peptides localize intracellularly, supporting our hypothesis that GP₂-derived peptides require intracellular localization to be effective in vivo. Similar results were obtained with influenza HA derived peptides 11 indicating that the lipid moiety and TAT are major drivers of subcellular localization for these two viruses.

In summary, we showed that TAT sequence and the lipid moiety both promote efficient intracellular localization and in vivo efficacy for intracellular fusing viruses, and both in various combinations may be useful for coronaviruses. Scientific premise: the coronavirus entry pathway into target cells is promiscuous.

### EXAMPLE 2 Design of SARS-CoV HRC Antiviral Peptides

Design, Generate and Characterize Improved Inhibitory SARS-CoV-2 S Specific C-Peptides

We identified lipid-derivatized MERS-CoV-S-derived entry inhibitors that effectively block MERS (see FIGS. 6 and 7). C-peptide inhibitors are designed and optimized for efficacy vs. SARS-CoV-2. CPP sequences and lipid conjugation are both necessary in order for the peptides to achieve optimal intracellular localization and in vivo efficacy, given that fusion blockade occurs in the endosome. We provide experimental evidence for this hypothesis for influenza and Ebola. As discussed elsewhere, for coronavirus fusion can occur either at cell membrane or after intracellular localization. We test whether improving endosomal localization by adding the CPP and modifying the lipid moiety increases antiviral potency for SARS-CoV-2.

Sequence of the HRC Domain of the SARSCoV-2 S Protein

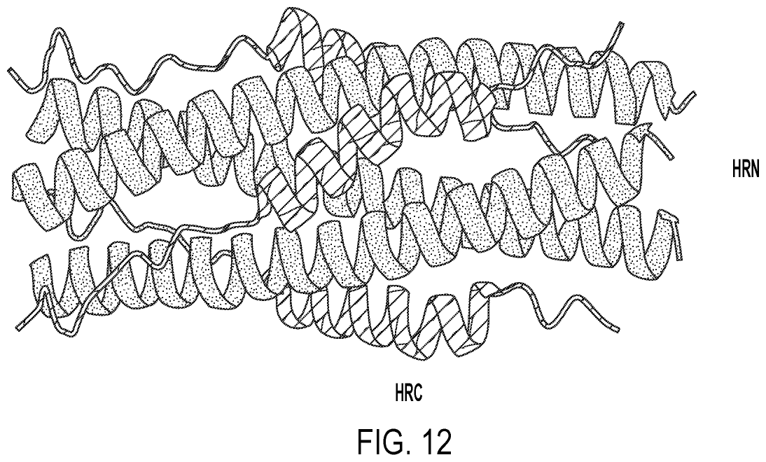

The SARS-CoV-2 6HB assembly (FIG. 12) provides an excellent basis for design of backbone-modified inhibitors of SARS-CoV-2 membrane fusion. The HRC domain features a central five-turn α-helix and extended regions flanking the helix on both sides. The native HRC domain corresponds to residues 1168-1203 of the SARS-CoV-2 S protein.

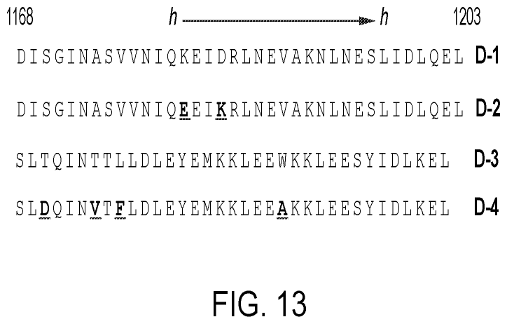

Peptide D-1 (FIG. 13) corresponds to the SARS-CoV-2 HRC domain (identical to the SARS-CoV-1 HRC domain); Xia et al. recently reported that D-1 is a modest inhibitor of SARS-CoV-2 infection in a pseudovirus-based cellular assay (IC₅₀˜1 μM). Residues that form the central α-helix are indicated. Proposed peptide D-2 contains two changes relative to D-1: Lys118toGlu and Asp1184toLys, which lead to an alternation of cationic and anionic side chains along the solvent-facing side of the helix. Therefore, D-2 should feature an array of ion pairs that stabilizes the helix23, and we predict that D-2 will be superior to D-1 as an inhibitor of SARS-CoV-2 infection. D-3 corresponds to the MERS S HRC domain, and D-4 is a derivative of D-3 that is comparable to D-1 as an inhibitor of SARS-CoV-2 infection.

A common approach will be used to assess HRC-based peptides generated in this program. Circular dichroism (CD) measurements will indicate whether HRC derivatives coassemble with the HRN peptide, and if so, to assess assembly stability. For promising HRC derivatives, cocrystallization with the HRN peptide will provide structures analogous to that in FIG. 12. Antiviral activity is initially assessed in cellular assays; promising candidates will be evaluated in human ex vivo and rodents in vivo assays.

Use Structure Guided Mutagenesis

TABLE 2
Lipid-conjugated Ebola GP₂-derived peptides
and their IC₅₀ against Zaire Ebolavirus in a
plaque reduction assay

| | Sequence and | | IC₅₀, μM |
|---|---|---|---|
| Name | modifications | MW | (95%CI) |
| TAT-EBO-dPEG4 | [symbol] | 5212 | >50 |
| TAT-EBO-PEG4- | [symbol] | 5827 | 0.05 |
| Chol | | | (0.029-0.071) |
| TAT-EBO-PEG4- | [symbol] | 5873 | 0.70 |
| Toco | | | (0.43-1.121) |
| TAT-EBO-PEG4- | [symbol] | 5827 | 0.09 |
| Chol | | | (0.055-0.153) |
| Chol-PEG4-TAT- | [symbol] | 5785 | 0.07 |
| EBO | | | (0.031-0.158)* |
| TAT-EBO-PEG24- | [symbol] | 6709 | 0.51 |
| Chol | | | (0.280-0.946) |
| Chol-PEG24-TAT- | [symbol] | 6667 | 3.04 |
| EBOV | | | (1.329-6.945)* |

[symbol]

indicates data missing or illegible when filed

Using structure-guided mutagenesis and protein engineering to optimize the antiviral potency and bioavailability of EBOV C-peptide fusion inhibitors, as an example to support the work for SARS-CoV-2 that we discuss here. We assessed the IC₅₀ of several peptides with modifications in the lipid moiety (either at the C or N terminal) and/or in the polyethylene glycol (PEG) spacer (size and origin). The results are shown in Table 2, above.

We also expanded the data in Table 3 using several strains of Ebola virus. The preliminary data show potency in the nanomolar range against several Ebola virus strains (see Table 3).

TABLE 3
Activity of lipid-conjugated GP₂ C-peptide
inhibitors against native Ebola viruses

| | ZEBOV | | ZEBOV | | Bundibugyo | |
|---|---|---|---|---|---|---|
| | Mayinga | | Gueckedou C07 | | EBOV | |
| | IC₅₀ | Max | IC₅₀ | Max | IC₅₀ | Max |
| Peptide | (μM) | % inh | (μM) | % inh | (μM) | % inh |
| TAT-EBO-PEG4-Chol | 0.05 | 97.9 | 0.02 | 97.5 | 0.07 | 98.5 |
| TAT-EBO-PEG4-Toco | 0.70 | 96.9 | 0.10 | 100 | 0.31 | 100 |

The newly identified sequence that we designed was tested against live virus (see Table 4, below). Based on biophysical data the sequence IEP (shown in highlight in Table 2) was modified to IAAILP highlight in Table 4). Contrary to our hypothesis, the TAT-EBO-IAAILP-PEG4-Chol (see table 4) had an IC₅₀ of 0.6 uM, around 10 times higher than both the TAT-EBO-PEG4-Chol and TAT-EBO-dPEG4-Chol (see Table 2) that have similar structures (PEG4 and cholesterol). We concluded that the sequence modification was detrimental to antiviral activity. However, we found that the TAT-EBO-IAAILP-Chol without PEG4 linker had an IC₅₀ of 3 nM, around 20 times better than the most potent peptide identified so far. Additionally, the IC90 was 27 nM—around half of the IC50 of our best peptide up to this point.

TABLE 4
Activity of modified
peptides against wild-type EBOV Mayinga

| | Sequence and | IC [symbol] | IC [symbol] |
|---|---|---|---|
| Peptide Name | modifications | (μM) | (μM) |
| TAT-EBO-IAAILP-Chol | [symbol] | 0.003 | 0.027 |
| TAT-EBO-IAAILP-PEG4- | [symbol] | 0.609 | 0.733 |
| Chol | | | |
| EBO-IAAILP-Chol | [symbol] | >10 | — |
| EBO-IAAILP-PEG4-Chol | [symbol] | >10 | — |

[symbol]

indicates data missing or illegible when filed

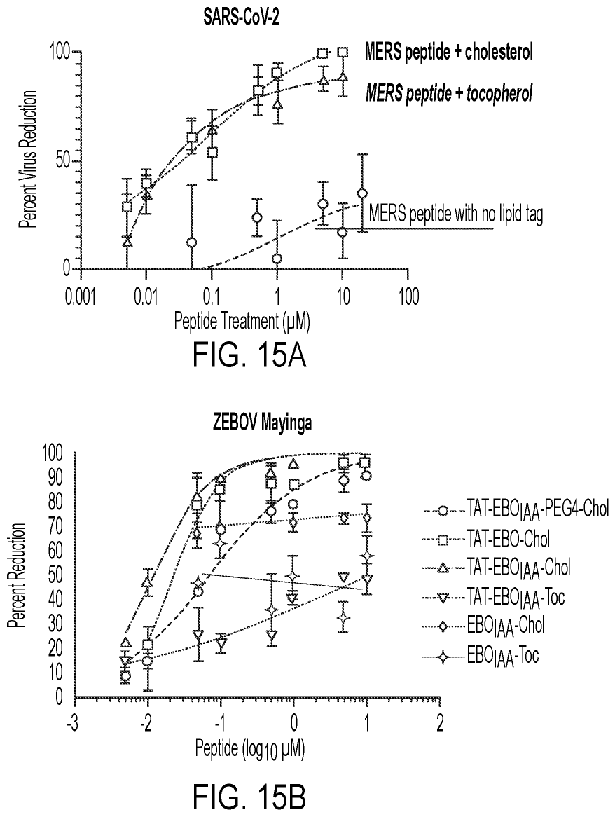

We prepared the TAT-EBO-Chol (the original sequence in Table 2 but without PEG). We tested the original sequence and compared it to the newly modified sequence in FIG. 15B. TAT-EBO-Chol without the PEG4 is significantly more potent than TAT-EBO-PEG4-Chol (see table 2), however the TAT-EBO-IAAILP-Chol without PEG4 linker is the most potent peptide we designed so far. The data show that both the sequence modification and PEG elimination are contributing to the increase in potency.

### EXAMPLE 3 Further Modification of SARS-CoV HRC Peptides

Addition of Lipid and Cell-Penetrating Peptide Sequences to Improve Efficacy and Intracellular Targeting

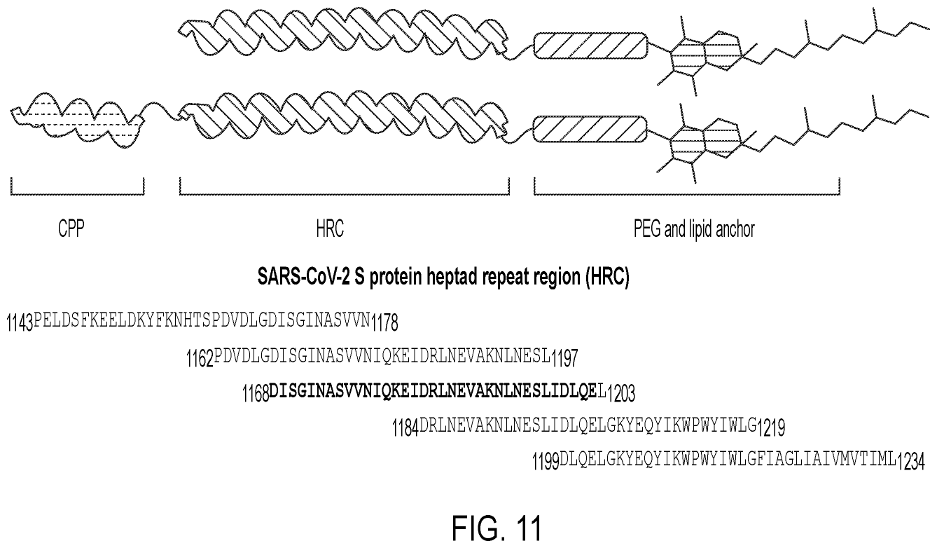

We designed SARS-CoV-2 S specific C-peptides (see FIG. 11). A total of 5 overlapping C-peptides (from the SARS-CoV-2 S HRC domain) will be synthesized (with and without cell penetrating peptide). The MERS sequence (with and without TAT) shown above to have broad spectrum activity 1 will be included. Another broad spectrum HRC derived sequence (EK1, with and without TAT) that inhibits SARS-CoV-2 fusion will be also included (this sequence has 5 aa differences from our MERS sequence). These 14 peptides (7 regular and 7 with TAT) will be initially conjugated with two lipids. The two lipids will be (i) cholesterol (since the most potent MERS peptide is a cholesterol conjugate, see FIG. 6) and (ii) tocopherol (since tocopherol conjugation, when combined with the TAT sequence, led to the most potent peptide in vivo for Ebola and influenza, Table 1). A PEG4 linker will be used (as in the peptides shown above in FIGS. 8 and 11). This set of 28 peptides (14 peptides X 2 lipids) be tested at CUIMC using a VSV pseudotyped virus based system (as in our work and fusion assay above). The most effective 10 peptides will be sent to UTMB for live SARS-CoV-2 testing (plaque reduction assay in Vero cells and confirmation in Calu-3 cells). The results of this preliminary screening will guide the selection of the 5 most potent peptides to advance to mechanistic study and broad spectrum evaluation. These 5 peptides will also advance to endosomal localization and ex vivo efficacy.

These data will provide information regarding the most effective HRC S derived aa sequence among 7 sequences (the 5 in FIG. 11, above, and the two MERS based peptides that have shown broad spectrum activity).

Assessment of peptide toxicity in monolayer cell culture: 5 peptides will be assessed for toxicity as in previous work⁵. Toxicity will be evaluated by Vybrant® MTT cell proliferation assay (Invitrogen).

### EXAMPLE 4 Assess Efficacy of HRC-Derived Peptides in Cell Culture

SARS-CoV-2 infections will be performed first in Vero cells with confirmation in Calu-3 cells, and peptides that show efficacy against live virus in these cells will move to experiments in HAE (commercially acquired). Serial dilutions of peptide inhibitors will be added either before or after infection to evaluate the effect of the peptides in preventing viral entry and whether the peptides block viral spread within the tissue after infection. In addition, we will study the HAE tissue for evidence of toxicity of the peptide using established protocols. We will use no more than ˜5 peptide inhibitors to study the ex vivo activity. We have shown that HAE are an ideal model to assess fusion inhibitory peptides activity. We have also recently shown that the human developmental lung organoid model represents the developing lung and can model several aspects of respiratory infections and we may use this model for SARS-CoV-2 in the future. These two models will be used as in our published work to assess peptides effectiveness against SARS-CoV-2. Viruses that emerge from growth in HAE (or organoids in future work) will be sequenced to assess evolution as we have done previously and to evaluate any peptide-resistant variants that emerge.

Middle East Respiratory Syndrome (MERS, caused by MERS-CoV) is a respiratory illness that was new to humans when it was first reported in 2012. We designed and produced several MERS-CoV specific lipid conjugated peptides based on a peptide sequence shown to be effective in vivo after intra-lung administration.

In 2014, these peptides designed by us were tested against MERS-CoV in fusion assays (FIG. 6) and in vivo (FIG. 7). These findings demonstrated that the MERS C-lipid-peptides are effective even after the entering viruses were targeted by the cellular proteases known to activate CoV spike-directed membrane fusion. The findings raise questions as to whether C-lipid-peptides must accumulate in endosomes to block CoV entry, and whether the spectrum of anti-CoV activity relates to requirements for proteolytic activation of CoV membrane fusion. Of note, there are new reports suggesting that serine protease inhibitors such as camostat and nafamostat may be useful inhibitors of SARS-CoV-2. As these serine protease inhibitors arrest or delay CoV fusion activation, they may synergize with the C-lipid-peptides that effect the same responses but in mechanistically distinct fashion.

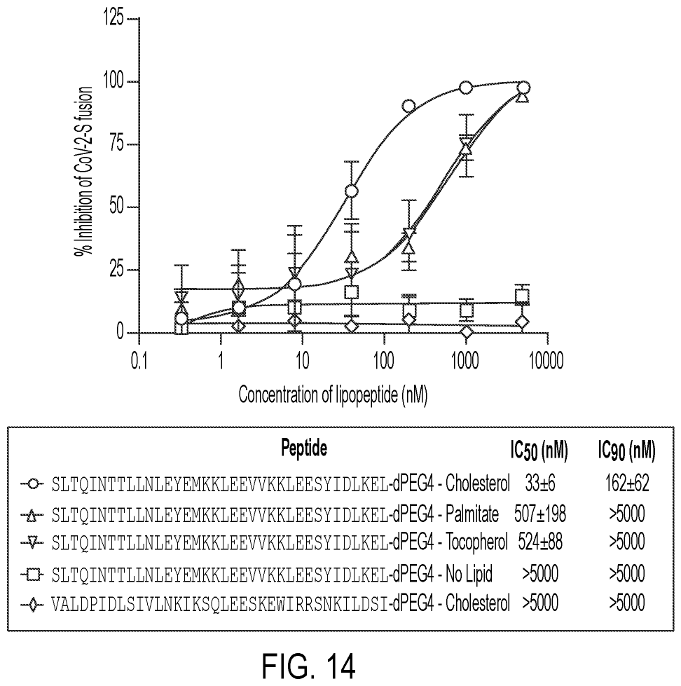

We have recently tested these MERS-S derived peptides in fusion assays using the SARS-CoV-2 S protein. Even without the cell penetrating sequence, the addition of lipid moieties increased the peptides' potency in fusion assays (FIG. 14). In that experiment, we found the best peptide (cholesterol conjugated) has an IC50 of around −33 nM, and IC90 of 200 nM. The IC50 and IC90 are better than our measles peptides in similar assays. These measles peptides can be given prophylactically and block infection in vivo when administered intranasally.

100% reduction in SARS-CoV-2 infection was observed using live virus and our MERS lipid-peptide in cell culture (FIG. 15A). In plaque reduction neutralization test in Vero-E6 cells, cells were infected with or without peptide and plaques were counted three days post infection. Results are expressed as percent reduction compared to virus not incubated with peptide. Values are means with standard deviations from triplicate wells. A similar setting was utilized to test the TAT-EBO-Chol fusions discussed above (Table 2-4), and the result is shown in FIG. 15B.

Lipid-peptide based on SARS-COV-2 was even more effective than the MERS lipid-peptides.

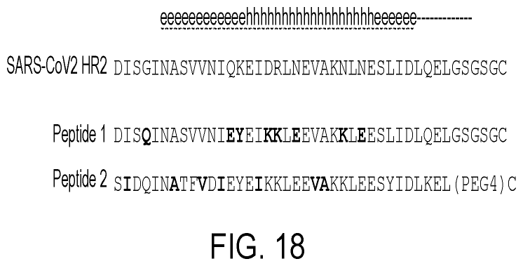

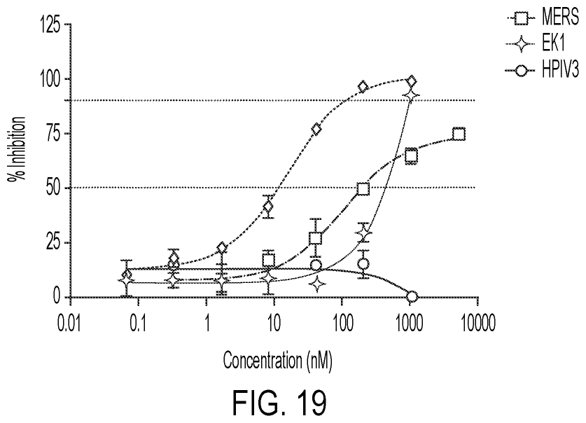

Inhibition of SARS CoV-2 glycoprotein fusion with the indicated peptides. The cell-to-cell fusion of 293T cells expressing SARS-CoV-2 glycoprotein bearing the indicated mutations and α-subunit of β-galactosidase with 293T cells transfected with ω-subunit of α-galactosidase and A) transfected hACE2 receptor or B) without transfected hACE2 receptor was assessed by a β-Gal complementation assay, in the presence of increasing concentrations of the indicated peptides. Resulting luminescence from β-galactosidase was quantified using a Tecan Infinite M1000 Pro. The percent inhibition of fusion (compared to results for control cells not treated with peptide) is shown as a function of the concentration of peptide. The values are means (±SD) of results from one experiment. The sequences of the peptides are in the diagram below (FIG. 18).

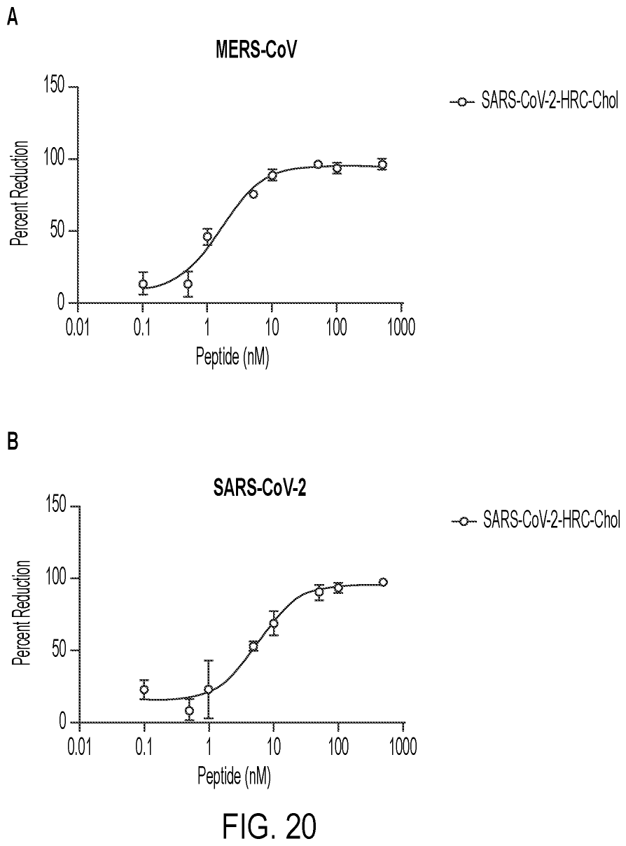

SARS lipid-peptides were effective against SARS live virus. IC50 is estimated at around 5-10 nM, indicating that the level needed is achievable in people. Notably, FIG. 20 shows that MERS and SARS virus are both inhibited by the prototype SARS peptide

### EXAMPLE 5 Ex Vivo Antiviral Activity and Virus Evolution Experiments to Study the Molecular Basis for Antiviral Activity and Resistance to C-Peptide Fusion Inhibitors

In order to understand the determinants of infection in the natural host, we will use the HAE model that has been used to characterize the polarity and cell specificity. We used this model for parainfluenza infection, confirming that it reflects virus-HAE interactions in the human lung. We and others have documented that results in immortalized monolayer cells may not be applicable when translated in vivo, and thus it is important to test our hypotheses in models that more closely represents the natural host. The HAE is ideal for assessing field isolates in experiments that replicate the clinical scenario.

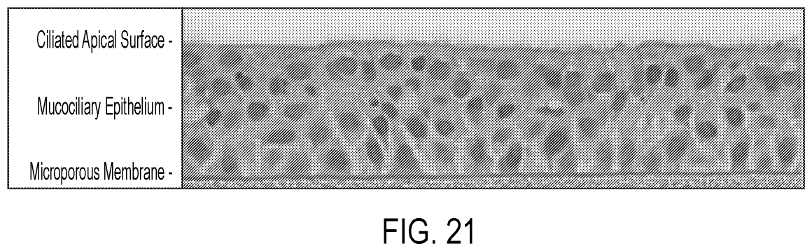

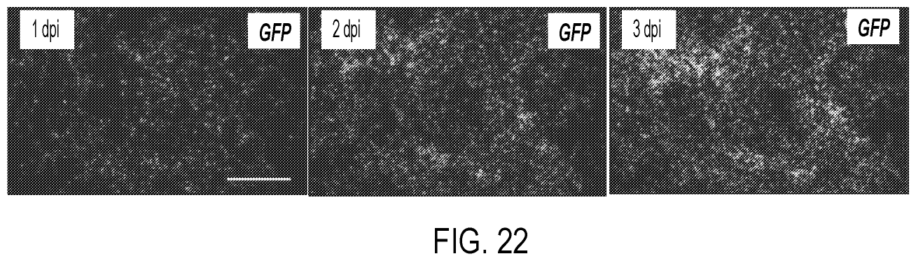

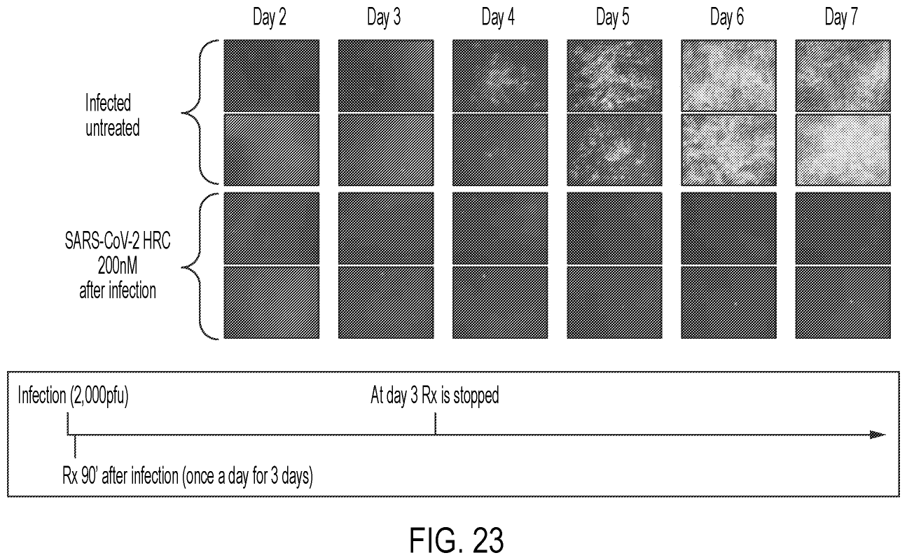

The human airway epithelium (HAE) mostly consists of large airway tissue grown at air-liquid interface (FIG. 21). We have validated human lung model for authentic viral growth and antiviral assessment for other viruses, including parainfluenza virus (FIG. 22). For the SARS-CoV-2 experiment The HAE was infected with SARS-CoV-2 bearing EGFP to visualize the infection (FIG. 23). Control tissues are not treated, the therapy tissues were treated with SARS-CoV-2 HRC at 200 nm, after the onset of infection. The HRC treatment effectively removed the infection.

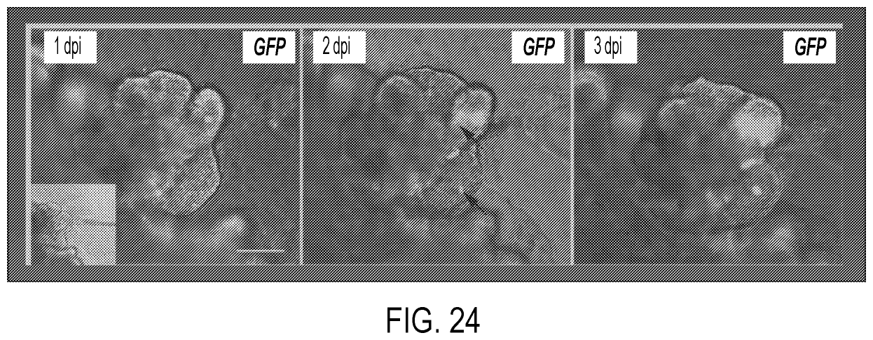

We also may utilize Human lung organoids as a model (FIG. 24). Both human lung ex-vivo models validated for authentic viral growth and antiviral assessment. (HAE validated for SARS-CoV-2 already, organoid has been validated for RSV, influenza, parainfluenza as in figure, and will be tested with SARS-CoV-2.)

The clinical use of Fuzeon© for HIV-1 resulted in the emergence of drug resistant HIV-1 variants. Escape variant viruses also emerged upon in vitro passaging of HIV-1 in the presence of Fuzeon©. The resistant viral population acquired mutations within a highly conserved stretch of three HRN amino acids, glycine-isoleucinevaline (GIV). Resistance mutations in this GIV motif also exist within the viral quasi-species of patients on Fuzeon© therapy. The resistance was due to either decreased interaction between the viral HRN and Fuzeon©, or increased interaction between viral HRN and HRC. Increased kinetics of fusion led to resistance, but also to viruses whose growth depended on the drug. While anti-SARS CoV-2 therapy will be of shorter duration than that for HIV (acute vs. chronic treatment), resistance may be important clinically, as it is for influenza. Based on the results in HIV and influenza, the in vitro data on emergence of resistance will apply directly to in vivo behavior of the viruses under selective pressure of treatment, and can be used to predict resistance and preemptively improve C-peptide fusion inhibitor design.

Strategy: SARS-CoV-2 infections will be performed in HAE. At recombinant SARS-CoV-2 virus bearing the EGFP gene (EGFP-SARS-CoV-2) has been recently produced. This virus will be used to monitor viral evolution under C-peptides' selective pressure in real time. Serial dilutions of peptide inhibitors will be added either before or after infection to evaluate (i) the effect of the peptides in preventing viral entry; (ii) whether the peptides block viral spread within the tissue after infection. In addition, we will study the HAE and organoid tissue for evidence of toxicity of the peptide using established protocols. Following assessment of antiviral activity in HAE, infections will be performed under the selective pressure of optimized C-peptide fusion inhibitors to analyze the molecular basis of potential resistance; to predict the possibility of evolution of C-peptide-resistant viruses; and to provide information that will be used to identify the C-peptide fusion inhibitors least likely to select for resistance.

Ex vivo antiviral activity: We have shown that HAE are an ideal model to assess fusion inhibitory peptides activity. This model is used to assess C-peptides effectiveness against SARSCoV-2, as in FIG. 23. Assessment in these models permits us to determine, in valid human ex vivo model (HAE), whether endosomal localization is beneficial for SARS-CoV-2 antiviral activity. Supernatant fluids from HAE will be divided in two aliquots, and processed for qPCR/genome sequence analysis and viral titer.

Generation of resistant variants: We will attempt to elicit SARS-CoV-2 viruses resistant to the inhibitory effect of small molecules using protocols routinely performed in our laboratory. Briefly, several dilutions of SARSCoV-2 will be passaged in HAE in the presence of several concentrations of C-peptides (ranging between 5x and 40x the IC50) for three to four days in HAE. Note C-peptides will be added after the initial infection to allow the viral polymerase complex to replicate and produce phenotypic variants for selection. Resistant viruses would spread even in the presence of C-peptides. Yields of virus will be determined by plaque assays and/or by qRTPCR. As the virus spreads in the presence and absence of inhibitor, the concentration of the inhibitor will be gradually increased to obtain a population of resistant viruses. Passaged virus will be sequenced as well as tested for inhibitor sensitivity in a plaque reduction assay. This strategy of applying selective pressure for viral evolution is similar to the informative experiments performed in our lab for neuraminidase-resistant variants and small molecule inhibitor-resistant variants.

Analysis of resistant variants in vitro: Mutant resistant viruses before and expansion (by growth in HAE), will be sequenced. We will analyze resistant virus mutants by high depth, whole viral genome sequencing. Sequences of the HAE-grown viruses will be compared to population-derived sequences generated during the duration of the selection experiments using custom bioinformatics software specifically made for longitudinal analysis of viral evolution. This approach will prevent us from neglecting potentially important viral subpopulations or alleles present across the genome that may co-exist during or after the selection process. We will determine whether the fitness of each variant is similar to that of the parent virus, or whether the variants require the presence of inhibitor for viability. We have extensive experience and previously validated both approaches, showing that the allele frequencies match for the two approaches with Paramyxoviridae such as canine distemper virus, human parainfluenza virus 3 (HPIV3), and respiratory syncytial virus. Shotgun sequencing enables a simple, one-workflow protocol for all RNA viruses, while tiling RT-PCR enables specific selection of viral sequences from complex sample types. We will sequence these viruses to a minimum average depth of 200X and call all variants with an allele frequency >4%. Sequence reads will be analyzed using our custom bioinformatic pipeline for longitudinal analysis of viral alleles in which reads for each sample are aligned to a de novo assembly consensus reference of the day/passage 0 viral genome.

If S contains mutations, we will introduce the mutated genes into our expression vectors and evaluate the glycoprotein functions in our functional assays. If multiple mutations are found, site-specific mutagenesis will be used to introduce the mutations into the S background, and singly-mutated genes will be analyzed for their phenotypes using the same in vitro assays. Location and conservation of the mutations will tell us the extent to which the resistance mechanism(s) for different peptides are similar. If the mutants derived from different peptides are markedly different, we will analyze the contributions of the specific mutations to dissect each contribution.

Analysis of resistant variants in vivo: If we identify resistant variants that grow well in vitro and ex vivo, we will assess their in vivo fitness. Resistant variants' pathogenicity will be compared in vivo to the parent virus. The total number of animals will depend on the number of resistant variants. One mouse model we will use to assess the resistant variants here and the peptides' efficacy is a human angiotensin-converting enzyme 2 (ACE2) transgenic mouse (hACE2 mouse). This model has been shown to be a lethal model for SARS-CoV-1. A recent report shows that for SARS-CoV-2 the model is not lethal, but weight loss and pathological signs are observed. Both gross pathology and histopathology can be easily observed at both day 3 and day 5 post infection. Viral titers of 10⁶-10⁷ pfu/ml were obtained after 1-3 days post infection. We have pre-ordered these mice from Jax laboratory and we expect to get mice in June 2020. This animal model of infection will be used to assess whether peptide-resistant variants cause altered pathology with respect to wt and if their fitness decreases or increases as a consequence of resistance mutations. The hACE2 mice will be used for assessment of antiviral efficacy. We anticipate testing ˜4 variant viruses plus 1 wt for a total of 5 viruses (10 animals; 5 males and 5 female-per group, for a total n=50 mice).

Sample collection and analysis: Tissue samples of all major organs will be collected from each mouse for histopathology assessment and viral load (by qRT-PCR). Virus isolation will be done only from specimens positive for EGFP-SARS-CoV-2 by qRT-PCR. Virus titration will be performed by plaque assay. Samples will also be sequenced to assess viral evolution in vivo.

The sequence alterations in S will be linked with the functional alterations, and this information will be used to understand resistance mechanisms. For parainfluenza, enhanced fusion kinetics led to partial resistance to peptide inhibitors in vitro, but we showed that mutations that increase fusion kinetics have a negative impact on growth in natural host tissue; these resistance mutations are likely to significantly reduce fitness in vivo. We expect the resistant CoV variants to be less pathogenic in vivo. We propose that by assessing these mechanisms early on in antiviral development we will avoid advancing antiviral strategies that could lead to more pathogenic viruses. Determining the ease of generation of variants and the fitness of SARS-CoV-2 containing resistance mutations will permit us to predict the likelihood of evolution of clinically relevant resistant variants. If resistance is acquired within four to five passages, we will consider combining our C-peptides with the protease inhibitors discussed elsewhere, to test the hypothesis that the combination treatment will provide a higher barrier to resistance. We also consider the possibility that no resistance will be elicited —although this is unlikely—, it would suggest that the fitness cost is too high to generate a viable variant resistant to that specific C-peptide. Such a C-peptide would be an ideal candidate to move forward. The information gained here will be used to select C-peptides with the lowest likelihood of eliciting resistance. The C-peptides that are effective in ex vivo, and are the least likely to elicit resistance in vivo, will be tested for in vivo efficacy.

Discussion: The primary focus is to obtain an effective C-peptide for the SARS-CoV-2 virus, but at the same time from these experiments we will know whether the coronavirus family can be inhibited by one C-peptide. We have already shown (FIG. 20) that one peptide inhibits both SARS-CoV-2 and MERS. Since the HRC of SARS-CoV-1 is the same of SARS-CoV-2 we expect the peptides to work for SARS-CoV-1 as well. We contend that this suggests that we will obtain broad spectrum coronavirus inhibitors. We will identify highly effective anti-coronavirus peptides for use in outbreaks and for stockpiling. We will obtain a detailed understanding of the molecular basis for the inhibitory activity of the C-peptides and address the basis for optimal inhibition. These results will reveal the correlation between structural and stability properties and inhibitory potency, and will be used to guide peptide design. The determinants of peptide localization inside the cells will be identified and leveraged to enhance the peptides' endosomal localization. A strength of our application is that the specific properties of the improved fusion inhibitors will be tested by assessing in vivo efficacy. The results will allow us to use our understanding of the biochemical and structural factors and optimal formulations that support efficacy, endosomal targeting and absent toxicity in order to design an ideal antiviral compound. We expect to obtain information on the molecular basis of resistance. The sequence alterations in resistant mutants will be linked with the functional alterations, and this information used to understand resistance mechanisms. The resistance studies, and in particular understanding the mechanisms of resistance, will provide insight into basic coronavirus fusion biology.

### EXAMPLE 6 Assessment of In Vivo Potency of Peptide Fusion Inhibitors in a Mouse Model of SARS-CoV-2 Infection

We will conduct pharmacokinetic and safety studies in mice. We will determine whether the in vitro improved peptides identified have the desired serum half-life and tissue biodistribution profiles, and whether they are safe and well tolerated in vivo. We will use the human angiotensin-converting enzyme 2 (ACE2) transgenic mouse50-52 (hACE2 mouse) to assess in vivo anti-SARS-CoV-2 efficacy. A recent report shows that for SARS-CoV-2 the model is not lethal, but weight loss and pathological signs are observed.

(https://www.biorxiv.org/content/10.1101/2020.02.07.939389v3). Both gross pathology and histopathology can be easily observed at both day 3 and day 5 post infection. Viral titers of 106-107 pfu/ml are obtained after 1-3 days post infection.

We showed that lipid modification not only increases the antiviral efficacy of the lead SARS-Cov-2 fusion-inhibitory peptide but also overcomes the typically poor pharmacokinetics of peptide drugs, prolonging the peptides' circulatory half-life to clinically useful levels. We will determine the extent to which the mutations and backbone modifications designed to increase anti-SARS-Cov-2 potency and protease resistance affect the pharmacokinetic properties of select improved SARS-Cov-2 peptides. Our goal is to ensure that the improved peptides reach an effective concentration in vivo, and to evaluate (i) the minimal dosage and (ii) frequency of administration required to maintain it. Here we also assess potential side effects and the kinetics of drug clearance. It is encouraging that in our in vivo paramyxovirus experiments and in preliminary pharmacokinetic studies no toxic effects were observed in mice and hamsters treated for up to 21 days at 20 mg/kg.

Pharmacokinetics (PK) of select improved peptides in mice (or in any other animal model that we find advantageous, as discussed below) will be assessed as we have previously done for similar peptide inhibitors. We will assess 4 peptides. Mice (6 per group, 3 males+3 females to capture sex as a variable) will be injected subcutaneously (s.q.), intraperitoneally (i.p.), intranasally (i.n), and (i.t) (we will initially test all four routes). Our preliminary data indicate that i.p. delivery is effective for MERS treatment (see FIG. 7). We will now compare i.p. with s.q., i.n. and i.t. since the three latter delivery routes would be preferable for clinical applications (i.n would be preferable for prophylaxis outside medical settings, while s.q./i.t. administration can be used in ill patients or those who cannot tolerate i.n. medications). Animals will be inoculated with fusion inhibitory peptide (6 mg/kg) and sacrificed 12, 24, 36 and 48 hrs later. We will perform ELISA for biodistribution studies and immunofluorescence. Evaluation of SARS-CoV-2 HRC peptide toxicity in mice: Acute, 15-day, and chronic toxicity.

Our published data show both prophylactic and therapeutic efficacy of lipidated peptides in vivo for Nipah virus, measles virus (MV), and influenza, and preliminary data for Ebola (not shown here) and MERS (FIG. 7) also show efficacy of lipidated peptides in vivo. The proposed challenge experiments will determine the minimum required dose for prophylactic efficacy and the therapeutic time window for peptide inhibitor efficacy. The mouse model we will use to assess efficacy is a hACE2 mouse50-52 described elsewhere (https://www.biorxiv.org/content/10.1101/2020.02.07.939389v3). This animal model of infection is ideal for assessment of peptide efficacy vs. viral replication and spread. Other animal models will be included or substituted as necessary.

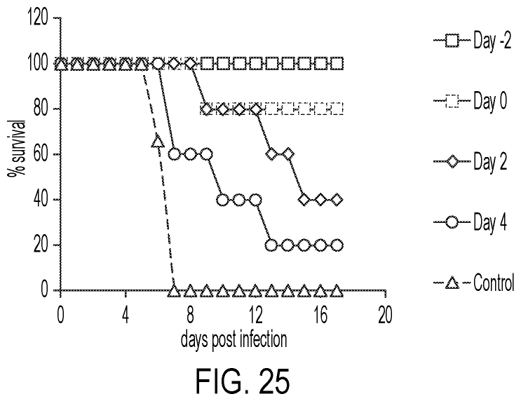

In vivo efficacy vs. Nipah (lethal virus) infection in golden hamsters: 2 mg/kg/d subcutaneous delivery of the lipid-peptide was effective (FIG. 25).

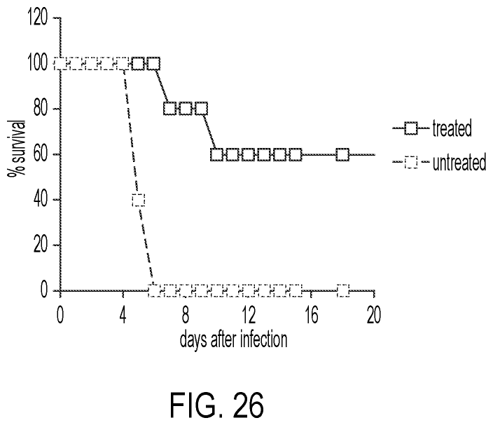

In vivo efficacy vs. Nipah (lethal virus) infection in golden hamsters: the lipid-peptide was administered intranasally. An administration at 1 day before, day of, 1 day after can provide 60% protection from lethal infection (FIG. 26).

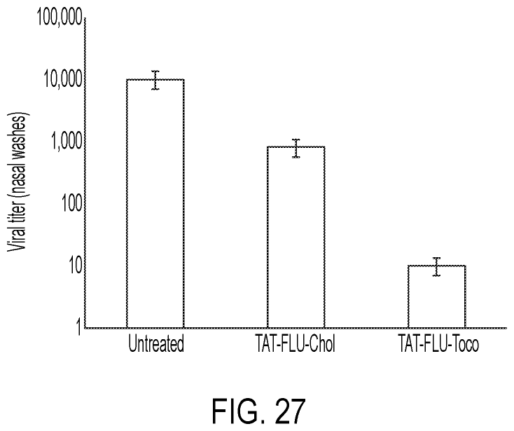

In vivo efficacy vs. influenza infection. Peptides given intranasally three times:1 day before, day of, 1 day after 1000x lower viral titer in cotton rats (FIG. 27).

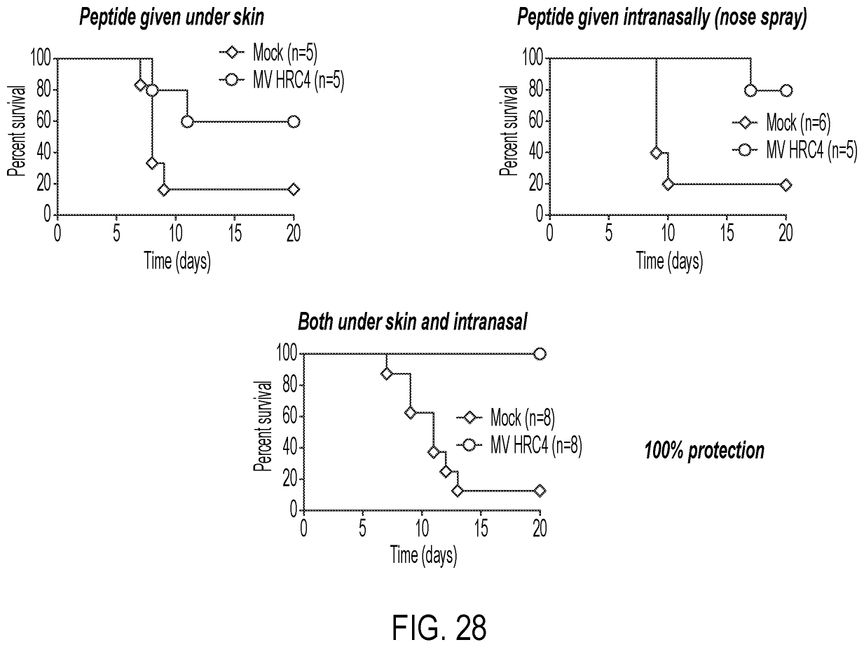

In vivo efficacy for preventing measles infection (fatal encephalitis) in mice with measles peptides. Both subcutaneous and intranasal administration were explored (FIG. 28).

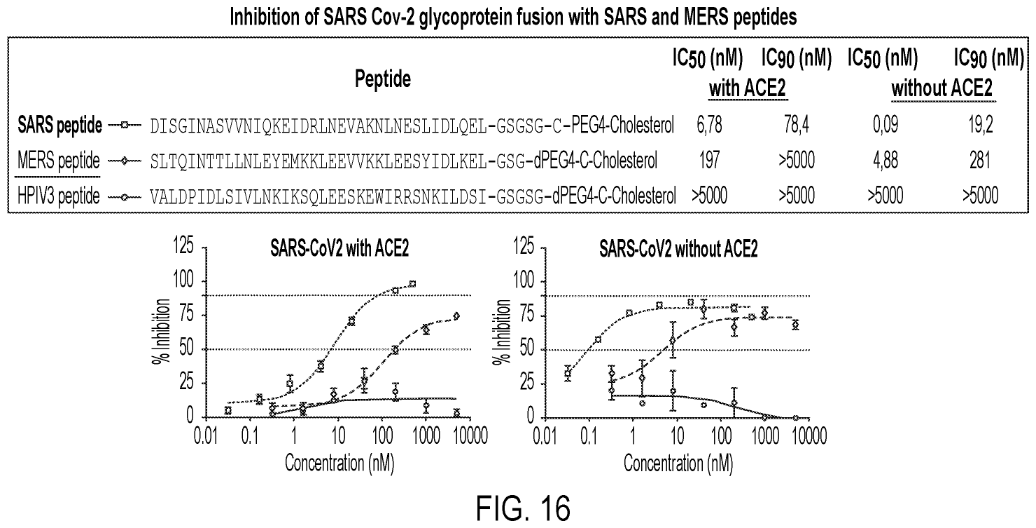

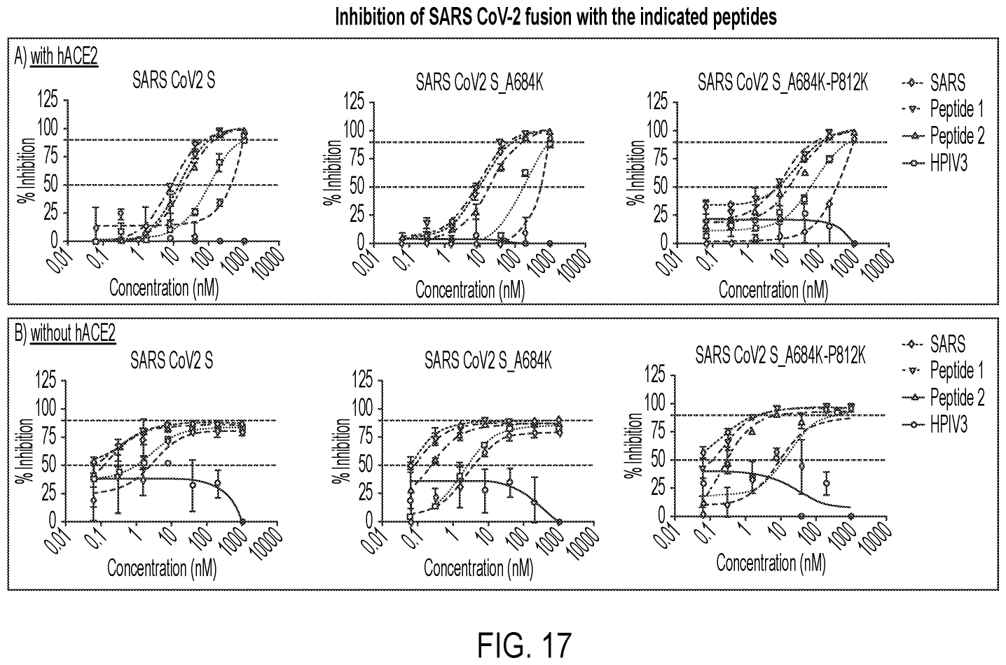

Comparison of quantitative fusion assay, with different expression level of hACE2 receptor. These data (shown above in FIG. 16) indicate that the peptides will be very effective in nasal/upper airway where there is less ACE2.

The most effective two peptide fusion inhibitors in vitro with favorable toxicity/biodistribution profiles will be tested in SARS-CoV-2 infections in hACE2 mice, and/or in the other relevant animal models as these become necessary and advantageous. We will first determine whether protection is afforded by i.n. peptide administration, prior to, concomitant with, or up to 10 days after the infection, and will establish the optimal dosage. Based on these data from we will undertake prophylactic and therapeutic studies using alternative delivery routes (s.q.).

Dosing: For the initial screening of two optimized peptide inhibitors, and for determining the optimal dose, groups of 10 animals will be treated with 3 different doses of the peptide i.n. and s.q. one day prior to challenge and then daily for up to 2 days. Infection will be performed with 105 TCID50 of SARS-CoV-2 i.n.

Efficacy: Once we determine an effective dose, we will focus on determining the therapeutic window for postexposure treatment. This is important, as this is an important likely use of the product to manage an outbreak. We will determine how many days after infection a peptide treatment can provide protection. See VA section.

Animal numbers: For dosing: (2 peptides+scrambled+mock treated)×10 mice X 2 inoculation routes X 3 doses=240 mice. Efficacy: 2 peptides X 10 mice X 1 inoculation route X 4 time points=80+10 untreated mice.

Viral load from lung will be determined by plaque assay and qRT-PCR. Sample tissues from treated and untreated animals will also be sent for sequencing to determine whether viral evolution occurred during treatment.

The readout of the model is clear, and statistically significant groups will be formed. Animals of both sexes will be used to ensure the capture of sex as a variable. We expect that prophylactic administration of the peptides will protect against infection. Whether the peptides are also effective in a post-exposure regimen will be determined. Intranasal delivery will likely work well for prophylaxis but it is possible that s.q. will work better after initial infection, and depending on the results we may decide to treat post-exposure via s.q. injection.

Quantitation of peptide concentration is done by ELISA, since we found that HPLC-MS has a limit of detection too high for assessment of peptides in certain organs (data not shown). Block amphiphiles such as our peptides could have surfactant properties, leading to epithelial irritation; therefore, testing toxicity is important. Only peptides that do not exhibit toxic effects will progress to the efficacy study. Therapy for coronaviruses is expected to be of short duration; however, it is possible that antibodies may be generated against peptides during treatment that may affect the treatment. We have not observed such an effect while studying Nipah-infected hamsters, measles infected mice and cotton rats. To test for the possibility of antibody antagonism to treatment, we will collect the serum from animals used for toxicity studies outlined above and assess for interference with the peptide's inhibitory activity. We consider the possibility that the explanation for the non-lethalitiy of in vivo infection with SARS-CoV-2 in hACE mice may be due to the old age of the animals (6-11 months). We will determine whether younger mice may permit a lethal model of infection. Survival curves would be a more statistically significant read out for efficacy.

From these experiments we will know whether we have an effective inhibitor for SARS-CoV-2 for the currently circulating CoV (and whether coronavirus family viruses can be inhibited by one peptide). We will obtain a good understanding of the molecular basis for the inhibitory activity of the peptides and correlation between structural and stability properties and inhibitory potency, useful to guide peptide design. As the result of the proposed work, we are confident—based on the data presented here and our published data that an effective prophylactic regimen will be achieved. Developing a post-infection treatment is more difficult; however, prophylaxis itself will be critically important. Health care workers would benefit directly from our prophylactic approach since it could be easily administered (e.g., once a day i.n.) and is effective immediately and at least for 24-hours (vs. a longer-term vaccine strategy). At risk people will also benefit from such a prophylactic treatment. At the end of this project, we will have (1) identified SARS-CoV-2 peptide fusion inhibitors; (2) optimized their antiviral activity in vitro and ex vivo; (3) tested the efficacy of these novel fusion inhibitors in a relevant animal model.

### EXAMPLE 7 Analysis of In Vivo Biodistribution and Toxicity of SARS-CoV-2 HRC Peptide Fusion Inhibitors

We will determine the anti-SARS-CoV-2 potency, protease resistance, and the pharmacokinetic properties of the SARS-CoV-2 C-peptides. Our goal is to ensure that the HRC peptides reach an effective concentration in vivo, and to evaluate (i) the minimal dosage and (ii) frequency of administration required to maintain it. Here we also assess potential side effects and the kinetics of drug clearance.

Pharmacokinetics (PK) of the HRC peptides in mice will be assessed as we have previously done for similar peptide inhibitors. The intratracheally (i.t.) delivery in mice provides consistent results compared to i.n. delivery and this will help the biodistribution study and be used for prophylactic studies to represent delivery via airway if needed. We will assess 4 peptides. Mice (6 per group, 3 males+3 females to capture sex as a variable) will be injected subcutaneously (s.q.), intraperitoneally (i.p.), intranasally (i.n), and (i.t) (we will initially test all four routes). Our preliminary data indicate that i.p. delivery is effective for MERS treatment (see FIG. 7). We will now compare i.p. with s.q., i.n. and i.t. Animals will be inoculated with fusion inhibitory peptide (6 mg/kg) and sacrificed 12, 24, 36 and 48 hrs later. Serum and organs (lungs, liver, spleen, and brain) will be collected. The organs will be split and either frozen with cold isopentane on dry ice for immunofluorescence or homogenized for semi-quantitative ELISA analysis.

Immunofluorescence: Cryo-sections will be stained with specific rabbit anti-SARS-Cov-2 HRC antibody (that we will generate using a contractual company as we have done regularly). Tissue sections will be analyzed using confocal microscopy.

ELISA for biodistribution studies: Organs will be homogenized using a “BeadBug” homogenizer. Peptide concentration in tissue samples and serum will be determined as we have done before5,7,8. Standard curves will be established for the lead peptides, using the same ELISA conditions as for the test samples (note this is more sensitive than the LC/MS/MS we used previously).

Evaluation of SARS-CoV-2 C-peptide toxicity in mice: We will undertake acute systemic toxicity testing in mice (for the peptides with the best biodistribution profile) to evaluate the toxicity and dose tolerance of the improved SARS-CoV-2 peptides. Purpose-bred outbred mice (n=6 per group, 3 male and 3 female) will receive a single s.q. injection of 5, 20 or 200 mg/kg of fusion inhibitory C-peptide. Harlan isovolumetric saline will serve as the control. Animals will be closely monitored for survival and/or signs of distress. For 15-day toxicity study, animals will be inoculated s.q. with peptide (20 mg/kg) for 15 consecutive days, and monitored daily. For chronic toxicity, peptides will be administered s.q. and i.n. to mice (n=6 per group) twice weekly at a dose of 20 mg/kg for 30 (i.e., 8 inoculations) or 60 (i.e., 16 inoculations) days. On days 30 and 60, animals will be sacrificed for determination of body and organ weights and gross pathologic examination (as described; 5,7,52-54) as well as for histopathology. Statistical significance of the mean of the treated group compared with that of the control group will be analyzed by a one-way analysis of variance, followed by Dunnett's multiple comparison tests using the Prism program (Graphpad, San Diego). Differences will be considered statistically significant if p<0.05.

These experiments will determine the effective half-life for the therapeutic dose and the gross biodistribution of SARS-CoV-2 peptide inhibitors in mice, and whether (as we anticipate) the SARS-Cov-2 peptide fusion inhibitor is non-toxic at likely therapeutic doses. More potent inhibitors will allow for lower dosages. Only the peptides that do not exhibit toxicity will progress to the efficacy study. We consider the possibility that a combination of the different delivery routes (s.q., i.p., i.t., and i.n.) may yield a favorable balance between biodistribution profile and ease of use with minimal adverse side-effects. Delivery to mucosal surfaces (e.g. i.n./i.t.) would be an easy and effective way to treat prophylactically, and this strategy would be applicable in the field or in hospitals (e.g. to protect health care providers). For critically ill patients or others who cannot tolerate i.n. medication, parenteral administration will be preferable. We expect to show that i.n. with a large volume (i.e., 50 μl) will result in consistent lung delivery 10 and this will be assessed by comparison to i.t., since i.t. has been shown to mimic delivery via airway. This will be important for in vivo challenge since (especially for prophylaxis) all animals should receive consistent dosage via i.n. In case i.n does not consistently result in distribution similar to i.t. we will consider i.t., at least for single prophylactic doses. From this we will select the peptides based on the longest biodistribution in the lungs.

Peptide immunogenicity studies: Measurement of antibodies associated with administration of peptides will be performed when conducting repeated dose toxicity studies. Anti-peptide antisera will be used to assay for antibodies generated during the chronic toxicity studies described above. We will attempt to evaluate effects of antibody responses on pharmacokinetics, incidence and/or severity of adverse effects, complement activation, or pathological changes related to immune complex formation and deposition.

### EXAMPLE 8 Ferret Models

Beyond mouse, we will also use different animal models as these are elucidated and become available. (www.sciencemag.org/news/2020/04/mice-hamsters-ferrets-monkeys-which-lab-animals-can-help-defeat-new-coronavirus)

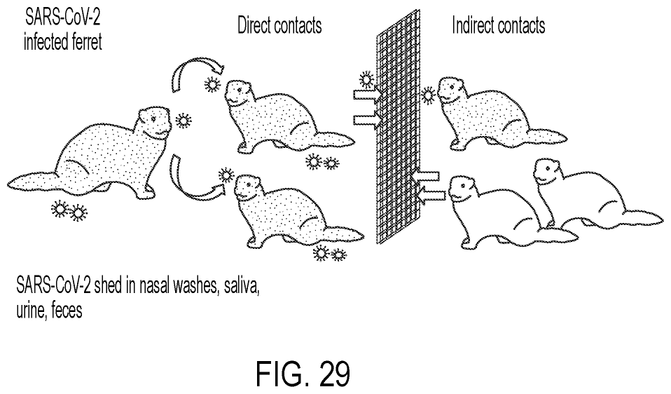

The first animal model we will use is the ferret (Kim et al., Infection and Rapid Transmission of SARS-CoV-2 in Ferrets, Cell Host & Microbe (2020)) for assessing whether our prototype peptide prevents direct transmission of SARS-CoV-2 from an infected animal to uninfected direct contacts. Ferrets are an ideal model for studying prophylaxis and transmission. This animal transmits SARS-CoV-2 very readily to uninfected ferrets, either by direct contact or from cage to cage. (Kim et al.) Ferrets will be treated with nose drops and assessed for protection from infection during contact with SARS-CoV-2 infected contact animals. All direct contacts become infected by 2 days. Ferrets will be treated with nose drops and assessed for protection from infection during contact with SARS-CoV-2 infected contact animals (FIG. 29).

### EXAMPLE 9 Human Trials

We look towards human safety/efficacy in health care workers and other first responders first, after permissible results can be gained from animal tests.

### SEQUENCE LISTING

(wild type SARS-CoV-2-HRC)
SEQ ID NO: 1
DISGINASVVNIQKEIDRLNEVAKNLNESLIDLQEL
(Peptide 1, modified SARS-CoV-2-HRC)
SEQ ID NO: 2
DISQINASVVNIEYEIKKLEEVAKKLEESLIDLQEL
(Peptide 2, modified SARS-CoV-2-HRC)
SEQ ID NO: 3
SIDQINATFVDIEYEIKKLEEVAKKLEESYIDLKEL
(Peptide 4, derived from Ebola virus GP₂)
SEQ ID NO: 4
IEPHDWTKNITDKIDQIIHDFVDK
(Peptide 5, derived from Ebola virus GP₂)
SEQ ID NO: 4
IAALPHDWTKNITDKIDQIIHDFVDK

What is claimed is:

1. A peptide comprising a sequence selected from the group consisting of SEQ ID NO:2, SEQ ID NO:3, SEQ ID NO:4, and SEQ ID NO:5.

2. A peptide comprising a sequence with more than 80%, 85%, 90%, 95%, but less than 100% homology with a sequence selected from the group consisting of SEQ ID NO:1, SEQ ID NO:2, SEQ ID NO:3, SEQ ID NO:4, and SEQ ID NO:5.

3. A lipid-peptide fusion, comprising a peptide of claim 1 or 2 and a lipid tag.

4. The lipid-peptide fusion of claim 3, wherein the lipid tag is Cholesterol, Tocopherol, or Palmitate.

5. A lipid-peptide fusion inhibitor, comprising a peptide of claim 1 or 2, a lipid tag, and a spacer.

6. The lipid-peptide fusion inhibitor of claim 5, wherein the spacer comprises a polyethylene glycol (PEG).

7. The lipid-peptide fusion inhibitor of claim 6, wherein the spacer comprises PEG4.

8. The lipid-peptide fusion inhibitor of any one of claims 5 through 7, wherein the lipid tag is Cholesterol, Tocopherol, or Palmitate.

9. The lipid-peptide fusion inhibitor of any one of claims 5 through 8, further comprising a cell penetrating peptide sequence (CPP).

10. The lipid-peptide fusion inhibitor of claim 9, wherein the CPP is HIV-TAT.

11. A pharmaceutical composition comprising a peptide of claim 1 or 2 and a pharmaceutically acceptable excipient.

12. A pharmaceutical composition comprising a peptide of claim 1 or 2, a lipid tag, and a pharmaceutically acceptable excipient.

13. The pharmaceutical composition of claim 12, wherein the lipid tag is Cholesterol, Tocopherol, or Palmitate.

14. A pharmaceutical composition comprising a peptide of claim 1 or 2, a lipid tag, a spacer, and a pharmaceutically acceptable excipient.

15. The pharmaceutical composition of claim 14, wherein the spacer comprises a polyethylene glycol (PEG).

16. The pharmaceutical composition of claim 15, wherein the spacer comprises PEG4.

17. The pharmaceutical composition of any one of claims 14 through 16, wherein the lipid tag is Cholesterol, Tocopherol, or Palmitate.

18. The pharmaceutical composition of any one of claims 14 through 17, further comprising a cell penetrating peptide sequence (CPP).

19. The pharmaceutical composition of claim 18, wherein the CPP is HIV-TAT.

20. A SARS-COV-2 (COVID-19) antiviral composition, comprising a SARS-COV-2 (COVID-19) lipid-peptide fusion inhibitor comprising a peptide selected from the group consisting of SEQ ID NO:1, SEQ ID NO:2 and SEQ ID NO:3, a lipid tag, a spacer, a CPP, and a pharmaceutically acceptable excipient.

21. The SARS-COV-2 (COVID-19) antiviral composition of claim 20, wherein the peptide is selected from SEQ ID NO:2 and SEQ ID NO:3.

22. A method of treating COVID-19 comprising administering to a subject in need thereof an antiviral pharmaceutical composition comprising a peptide with more than 80% homology with a sequence selected from the group consisting of SEQ ID NO:1, SEQ ID NO:2 and SEQ ID NO:3, a lipid tag, a spacer, a CPP, and pharmaceutically acceptable excipients.

23. The method of claim 22, wherein the lipid tag is Cholesterol, Tocopherol, or Palmitate.

24. The method of claim 22 or 23, wherein the antiviral pharmaceutical composition is administered per airway or subcutaneously.

25. The method of claim 24, wherein the antiviral pharmaceutical composition is administered intranasally.

26. The method of claim 25, wherein the antiviral pharmaceutical composition is administered as nasal drops or a spray.

27. An Ebola antiviral composition, comprising an Ebola lipid-peptide fusion inhibitor comprising a peptide selected from SEQ ID NO:4 and SEQ ID NO:5, a lipid tag, a CPP, and a pharmaceutically acceptable excipient.

28. An Ebola antiviral composition of claim 27, further comprising a spacer.

29. A method of treating Ebola comprising administering to a subject in need thereof an antiviral pharmaceutical composition comprising a peptide with more than 80% homology with a sequence selected from SEQ ID NO:4 and SEQ ID NO:5, a lipid tag, a CPP, and pharmaceutically acceptable excipients.

30. The method of claim 29, wherein the peptide further comprises a spacer.

31. The method of claim 29 or 30, wherein the lipid tag is Cholesterol, Tocopherol, or Palmitate.

32. The method of claim 22 or 23, wherein the antiviral pharmaceutical composition is administered per airway or subcutaneously.
